(19) United States
(12) Patent Application Publication
(10) Pub. No.: US 2005/0037349 A1
(43) Pub. Date: Feb. 17, 2005

(54) Method for the preparation of reagents for amplification and/or detection of nucleic acids that exhibit no significant contamination by nucleic acids

(76) Inventors: Francois J. Picard, Quebec (CA); Christian Menard, Quebec (CA); Martine Bastien, Quebec (CA); Maurice Boissinot, Quebec (CA)

Correspondence Address: STERNE, KESSLER, GOLDSTEIN & FOX P.L.L.C., 1100 NEW YORK AVE., N.W., WASHINGTON, DC, 20005, US

(21) Appl. No.: 10/469,419

(22) PCT Filed: Apr. 11, 2003
(86) PCT No.: PCT/CA03/00548

Related U.S. Application Data
(60) Provisional application No. 60/371,428, filed on Apr. 11, 2002.

(51) Int. Cl. C12Q 1/68; C07H 21/04

(52) U.S. Cl. 435/6; 536/25.32

(57) ABSTRACT

The present invention decribes reagents free of detectable contaminating nucleic acids for performing highly sensitive and specific nucleic acids amplification and/or detection. It relates to an improvement in the technology of nucleic acid inactivation prior to nucleic acid testing (NAT) in order to prevent false-positive results. Specifically, this invention describes optimized and standardized reagents and ultra-violet treatment to achieve an effective and highly reproducible nucleic acid inactivation prior to NAT without substantially affecting the performance of the assay. More specifically, this nucleic acid inactivation process resulted in a reduction of up to four logs of the background signal associated with the PCR (polymerase chain reaction) amplification of DNA contaminating PCR reagents. This optimized and standardized method is also adaptable for use with NAT technologies other than PCR.

### FIELD OF THE INVENTION

The present invention relates to reagents submitted to an improved treatment using furocoumarin derivatives (e.g. psoralens and/or isopsoralens) and UV irradiation to inactivate contaminating DNA and/or RNA from nucleic acid testing (NAT) reagents, without or with minimal hindering of the performance of the NAT method.

### BACKGROUND OF THE INVENTION

The practical application of recombinant DNA technology in the field of infectious diseases was initially reported in 1980 by Moseley et al. (Moseley et al., 1980, J. Infect. Dis. 142:892-898). Since those days, molecular biology technologies have undertaken a rapid evolution. Based on these technologies, a number of rapid and sensitive nucleic acid testing (NAT) methods have been developed for a variety of applications including diagnosis of infectious and genetic diseases in humans, animals and plants. Many of these NAT assays have been used in the field of microbiology to complement or replace the slower conventional culture-based identification systems (Picard and Bergeron, 2002, Drug Discovery Today 7:1092-1101; Boissinot and Bergeron, 2002, Curr. Opinion Microbiol. 5:478482; Tang and Persing, 1999, Molecular detection and identification of microorganisms, p. 215-244, In Manual of Clinical Microbiology, Murray et al., American Society for Microbiology, Washington, D.C.; Lee et al. 1997, Nucleic Acid Amplification Technologies: Application to Disease Diagnosis, Biotechniques Books, Eaton Publishing, Boston, Mass.; Persing et al., 1993, Diagnostic Molecular Microbiology: Principles and Applications, American Society for Microbiology, Washington, D.C.). These assays have been designed for microbial detection and identification directly from clinical and/or environmental samples and are based on the use of a variety of NAT technologies including the widely used and powerful polymerase chain reaction (PCR). Other nucleic acid amplification technologies include among others the ligase chain reaction (LCR), the strand displacement amplification (SDA) as well as transcription-based amplifications such as the transcription mediated amplification (TMA) (Tang and Persing, 1999, Molecular detection and identification of microorganisms, p. 215-244, In Manual of Clinical Microbiology, Murray et al., American Society for Microbiology, Washington, D.C.; Lee et al., 1997, Nucleic Acid Amplification Technologies: Application to Disease Diagnosis, Biotechniques Books, Eaton Publishing, Boston, Mass.). Sensitive NAT technologies also include signal amplification methods such as the branched DNA (bDNA) probe technique.

NAT can be used to detect the presence of any microbe in clinical samples. A number of PCR-based assays targeting highly conserved nucleotide sequences in microbes have been used by us and others to develop universal amplification assays for bacteria or fungi (Martineau et al., 2001, J. Clin. Microbiol. 39:2541-2547; Schonhuber et al., 2001, BMC Microbiology 1:20; Ke et al., 1999, J. Clin. Microbiol. 37:3497-3503; Loeffler et al, J. Clin. Microbiol. 37:1200-1202; McCabe et al., 1999, Molecular Gen. Metabolism 66:205-211; Klausegger et al., 1999, J. Clin. Microbiol. 37:464-466; Tanner et al. 1998, Appl. Environ. Microbiol. 64:3110-3113; Goh et al., 1996, J. Clin. Microbiol. 34:818-823; Sandhu et al., 1995, J. Clin. Microbiol. 33:2913-2919; Greisen et al., 1994, J. Clin. Microbiol. 32:335-351; Schmidt et al., 1991, Biotechniques 11:176-177; Rand and Houck, 1990, Mol. Cell. Probes 4:445-450 and our co-pending patent application WO 01/23604 A2). However, because of the high sensitivity of NAT, the development of sensitive and broad-range (or universal) nucleic acid detection assays is hampered by the presence of microbial DNA and/or microbial cells that may be present in NAT reagents and which lead to false positive results.

The most common source of false-positive results in NAT is associated with carry-over of previously amplified target nucleic acids. This type of contamination can be prevented by using proper laboratory procedures (Millar et al., 2002, J. Clin. Microbiol. 40:1575-1580; Kwok and Higuchi, 1989, Nature, 239:237-238), or alternatively, by using techniques to inactivate amplification products such as the method using the uracil-N-glycosylase (UNG) (Longo et al., 1990, Gene 93:125-128). DNA inactivation using the photoreactive compounds psoralen or isopsoralen, which is used in the object of the present invention, may prevent amplification of contaminating target nucleic acids (Persing and Cimino, 1993, Amplification products inactivation methods p. 105-212, In Persing et al., Diagnostic Molecular Microbiology: Principles and Applications, American Society for Microbiology, Washington, D.C.; Isaacs et al., 1991, Nucleic Acids Res. 19:109-116; and U.S. Pat. No. 5,221,608). Psoralens and isopsoralens are furocoumarin compounds representing a class of planar tricyclic photoreactive reagents that are known to form covalent monoadducts and crosslinks with nucleic acids upon activation with ultra-violet (UV) light. Examples of furocoumarin compounds are given in U.S. Pat. No. 5,221,608, the contents of which are entirely incorporated by reference. These monoadducts can be formed between two adjacent pyrimidines on opposite strands of nucleic acids thereby creating interstrand crosslinks with both DNA and RNA. Such crosslinks prevent primer extension activities of polymerases. Psoralens and isopsoralens have the major advantage of allowing nucleic acid inactivation in closed vessels (such as PCR reaction vessels) thereby preventing carry-over contamination by nucleic acid aerosols. Another effective strategy to prevent carry-over contamination is to perform the nucleic acid amplification reactions in closed vessels such as in real-time PCR amplification and analysis (Foy and Parkes, 2001, Clin. Chem. 47:990-1000).

Another important source of false-positive results in NAT is extraneous nucleic acids introduced in reagents during the manufacturing process. For example, the Taq polymerase used in PCR has been shown by many investigators to be contaminated with bacterial DNA (Gale et al., 2003, Clin. Chem. 49:415-424; Corless et al., 2000, J. Clin. Microbiol. 38:1747-1752; Maiwald et al., 1994, Mol. Cell. Probes 8:11-14; Meier et al., 1993, J. Clin. Microbiol. 31:646652; Schmidt et al., 1991, Biotechniques 11:176-177; Jinno et al., 1990, Nucleic Acids Res. 18:6739; Rand and Houck, 1990, Mol. Cell. Probes 4:445-450; and U.S. Pat. No. 5,532,145). Analysis of the conserved bacterial rRNA gene sequences contaminating different preparations of Taq DNA polymerase revealed that these nucleic acids were closely related to the genera Corynebacterium, Afthrobacter, Mycobacteiium, Pseudomonas, Alcaligenes and Azotobacter (Hughes et al., 1994, J. Clin. Microbiol., 32:2007-2008; Maiwald et al., 1994, Mol. Cell. Probes 8:11-14). Importantly, the contaminating DNA sequences did not match with that of the species Eschedchia coli and Thermus aquaticus which were the bacteria used to produce these ezymes. Because of the nature of this type of contamination, the use of UNG or of closed vessel assays as well as careful laboratory techniques cannot circumvent this important NAT reagents nucleic acid contamination problem.

DNA inactivation using psoralens or isopsoralens combined with a UV treatment has been used to prevent amplification of microbial DNA contaminating PCR reagents (Corless et al., 2000, J. Clin. Microbiol. 38:1747-1752; Klausegger et al., 1999, J. Clin. Microbiol. 37:464-466; Hughes et al., 1994, J. Clin. Microbiol., 32:2007-2008; Meier et al., 1993, J. Clin. Microbiol. 31:646-652; Jinno et al., 1990, Nucleic Acids Res. 18:6739; and U.S. Pat. No. 5,532,145). However, there is no standardized method for nucleic acid inactivation using these photoreactive compounds allowing efficient and reproducible nucleic acid inactivation without substantial reduction in the performance of the nucleic acid amplification and/or detection assay. The words “without substantial” or “not have a substantial” are used throughout the present invention to mean “without or with minimal”.

Several investigators have reported an important reduction in the analytical sensitivity of NAT assays attributable to the UV treatment in the presence of psoralen or isopsoralen (Corless et al., 2000, J. Clin. Microbiol. 38:1747-1752; Meier et al., 1993, J. Clin. Microbiol. 31:646-652 and U.S. Pat. No. 5,532,145). Corless et al. (2000, J. Clin. Microbiol. 38:1747-1752) compared several methods to eliminate nucleic acid contamination from PCR reagents. They concluded that it was not possible to eliminate contaminating nucleic acids from the PCR reagents without significantly decreasing the analytical sensitivity of their real-time PCR assays. When they tested a combination of 8-methoxypsoralen (8-MOP) and UV irradiation, complete DNA decontamination of the PCR reagents was achieved after 5 minutes of UV exposure. They have not specified the UV dose (in mJoule/cm²) nor did they described the reagent container and its distance from the UV source. They observed a 5 to 7 logs reduction in the analytical sensitivity of the real-time PCR assays using this non-standardized 8-MOP-based DNA inactivation method. In fact, their experimental procedure does not include proper control of key parameters such as those disclosed in the present invention which ensure that an optimal UV energy dose is administered to the reagents containing an optimal 8-MOP concentration. The present invention allows for efficient nucleic acids inactivation while reducing the performance of the assay by only about 1 log or less. This is achieved by (i) monitoring the energy dose with a UV sensor by measuring the UV dose in mJoules per square centimeters, (ii) maintaining a constant distance between the reagents and the UV source, (iii) testing the reagent container for its permeability to UV treatment and (iv) optimising the 8-MOP concentration.

U.S. Pat. No. 5,532,145 describes the use of degassing to remove oxygen from PCR reaction mixtures containing a furocoumarin prior to UV irradiation to preserve Taq DNA polymerase activity. However, the degassing process is not practical as it involves freezing the reaction mixture to be decontaminated in dry/ice ethanol, thawing and applying vacuum for 30 seconds three times. As revealed in the present invention it is simpler to control the parameters of the UV treatment. These parameters include the type of furocoumarin compound and its concentration, the UV exposure, the intensity of the UV source, the length of the UV treatment and the wavelengths spectrum of the UV source which are important factors in achieving an efficient and reproducible performance in DNA inactivation, and this, without substantial detrimental effect on the performance of NAT assays. Other methods to inactivate DNA contaminating NAT reagents have been used with very limited success. These methods include the use of UV irradiation alone, a treatment with DNAase and/or restriction endonucleases and a treatment with exonucleases (Corless et al., 2000, J. Clin. Microbiol. 38:1747-1752; Zhu et al., 1991, Nucleic Acids Res. 19:2511). Also, a pre-filtration step for the PCR mix prior to the addition of the test sample have been used to remove nucleic acids present in PCR reagents (Yang et al., 2002, J. Clin. Microbiol. 40:3449-3454).

### SUMMARY OF THE INVENTION

The present invention relates to reagents submitted to an improved treatment using furocoumarin derivatives (e.g. psoralens and/or isopsoralens) and UV irradiation to inactivate contaminating nucleic acids from NAT reagents, without substantial hindering of the performance of the NAT methods, and this, without the need to remove oxygen in order to avoid the presence of damaging oxygen radical species (by degassing for example). This treatment includes careful control and monitoring of some experimental conditions including the quality of the vessel containing the reaction mixture to be treated as well as the UV dose and intensity of the light source in the UV wavelengths spectrum. The present method and resulting products (reagents and containers with reagents) ensure a reproducible and efficient nucleic acid inactivation.

It is an object of the present invention to provide reagents useful in the obtention of samples which are to be submitted to amplification and/or detection of nucleic acids in which the concentration of amplifiable and/or detectable contaminating nucleic acids is low, if not totally absent, so as not to substantially interfere with the detection of the nucleic acids targeted in the reaction.

These reagents may include a protein, the function of which should not be substantially affected by the treatment of this invention. Such a protein may be an enzyme. If a nucleic acid amplification reaction is to be performed, the enzyme may be a polymerase, a reverse transcriptase, a ligase or a restriction endonuclease. It may also be an enzyme useful in the test sample preparation steps for nucleic acid extraction preceding an amplification and/or detection reaction, for example a DNAase, a RNAase or a protease.

These reagents include nucleotides and/or nucleotide analogs, oligonucleotides (primers and/or probes), buffer solutions, ions (monovalent and/or divalent), enzymes (DNA polymerase, RNA polymerase, reverse transcriptase, DNA ligase, restriction enzymes, DNAase, RNAase, protease or any other enzymes used for NAT or in test sample preparation for NAT), amplification facilitators (e.g. betaine, dimethyl sulfoxide, bovine serum albumin, tetramethylamonium chloride), cryoprotectors (e.g. glycerol), stabilizers (e.g. trehalose) and a solvent (usually water). In a particularly preferred embodiment, these reagents containing no or a low level of detectable contaminating DNA or RNA may be provided separately or as separate components of a kit, or mixed together, and may be liquid, frozen or dehydrated. Preferably, the reagents are any combination suitable for a nucleic acid amplification and/or detection reaction.

It is another object of the present invention to provide for cleaner reagents and kits for the preparation of nucleic acids (sample preparation and nucleic acids extraction) for NAT assays as well as to provide an efficient method to inactivate nucleic acids contaminating said reagents and kits including purifying devices and columns.

It is another object of this invention to provide a container, such as a closed vessel, which comprises the reagents treated in accordance with the present invention. The closed vessel could be submitted to the same treatment, simultaneously with the treatment of the reagents. Indeed, the reagents could be placed into the vessel and then submitted to the treatment of this invention.

It is another object of this invention to provide an improved method using furocoumarin compounds and UV light for nucleic acid inactivation to treat reagents prior to NAT in order to prevent false-positive results, said improved method comprising:
A. A reaction mixture which contains reagents and enzymes required for NAT per se or for one or more preparative steps prior to NAT, as well as one or more furocoumarin compound(s); and
B. Said reaction mixture being treated with UV light under controlled conditions wherein the UV exposure as well as the intensity of the emission peaks of the light source in the UV spectrum are monitored to ensure a delivered UV dose sufficient to inactivate contaminating nucleic acids without substantial detrimental effect on the performance of the NAT assay;
For NAT assay, the following steps would be added:
C. Said UV treated reaction mixture being subsequently supplemented with the test sample and/or an internal control template; and
D. Said reaction mixture supplemented with the test sample and/or internal control template being subjected to nucleic acid testing per se under appropriate conditions. The testing preferably involves nucleic acids amplification and/or detection.

The furocoumarin compound is usually a psoralen or an isopsoralen derivative. In a preferred embodiment, the furocoumarin compound is 8-methoxypsoralen (8-MOP), trioxsalen, psoralen and/or FQ (1,4,6,8-tetramethyl-2H-furo[2,3-h]quinolin-2-one). In a particularly preferred embodiment, the furocoumarin compound is 8-MOP.

In a preferred embodiment, NAT is performed by using target or probe amplification techniques or signal amplification techniques or any other NAT technologies performed in liquid phase or onto solid supports. In a particularly preferred embodiment, NAT is performed by using the PCR amplification technology performed in liquid phase or onto solid supports.

In a preferred embodiment, the container wherein the NAT assay may take place is the immediate container in which the NAT is performed. It is usually a closed vessel. The closed vessel may also be a tubing or a tube. In a particularly preferred embodiment, the closed vessel is a plastic tube.

The UV treatment is performed using an apparatus consisting of a chamber equipped with a UV source and a UV sensor to monitor the energy dose of the treatment.

In a preferred embodiment, the intensity of the emission peaks of the light source in the UV spectrum is monitored using a UV sensor. In a particularly preferred embodiment, said UV sensor is used to monitor the intensity of the emission peaks of the light source in the UV spectrum inside the UV irradiation chamber of an apparatus.

In a preferred embodiment, the intensity of the emission peaks of the light source in the UV spectrum generated is monitored using a suitable radiometer or spectrometer. In a particularly preferred embodiment, said radiometer or spectrometer is used to monitor the intensity of the emission peaks of the light source in the UV spectrum inside the UV irradiation chamber of an apparatus.

The test sample may be of any origin, preferably of clinical or environmental source.

In another preferred embodiment, an internal control is used to verify the efficiency of each NAT reaction.

In another preferred embodiment, the detection method is based upon hybridization with a labelled probe. In a further preferred embodiment, the said probe is labelled with a fluorophore.

### DETAILED DESCRIPTION OF THE INVENTION

This invention will be described hereinbelow, with reference to specific or preferred embodiments and accompanying figures, the purpose of which is to illustrate the invention rather than to limit its scope.

### BRIEF DESCRIPTION OF THE FIGURES

FIG. 1: Examples of automated systems for manufacturing processes allowing controlled UV treatments and aliquoting of the treated reagents. Panel A: Manufacturing process using a tubing in which the reagent flow is controlled by a pump. The treated reagents are subsequently aliquoted in the NAT reaction vessels. Panel B: Manufacturing process using the immediate container in which the NAT is performed. This panel shows an example with the Smart Cycler tubes from Cepheid.

FIG. 2: UV irradiation chamber of the Spectrolinker apparatus. Panel A: Top view. Panel B: Side view.

FIG. 3: Determination of the optimal UV exposure for psoralen-based DNA inactivation of PCR reagents.

Melting curves of the PCR products amplified on a LightCycler with the Staphylococcus-specific PCR assay (Tₘ around 83° C.) showing the difference between different UV exposures. The peaks in the range of 70 to 82° C. correspond to the Tₘ of non-specific amplification products including primer dimers. Purified genomic DNA from Staphylococcus aureus ATCC 29737 (100 genome copies per reaction) was added to all reaction mixtures prior to DNA inactivation. Panel A: Melting curves after DNA inactivation with a UV dose of 1000 mJ/cm², Panel B: DNA inactivation with a UV dose of 1500 mJ/cm², Panel C: DNA inactivation with a UV dose-of 2000 mJ/cm², Panel D: DNA inactivation with a UV dose of 2400 mJ/cm² and Panel E: untreated reactions.

FIG. 4: Determination of the optimal psoralen concentration for DNA inactivation of PCR reagents.

Real-time detection on a Smart Cycler using a Streptococcus agalactiae-specific PCR assay showing the difference between different 8-MOP concentrations. Purified genomic DNA from Streptococcus agalactiae ATCC 12973 (10⁶ genome copies per reaction) was added to all reaction mixtures prior to DNA inactivation. Panel A: DNA inactivation with a 8-MOP concentration of 0.03 μg/μL, Panel B: DNA inactivation with a 8-MOP concentration of 0.06 μg/μL, Panel C: DNA inactivation with a 8-MOP concentration of 0.12 μg/μL and Panel D: DNA inactivation with a 8-MOP concentration of 0.24 μg/μL. The curve (Δ) of each panel corresponds to a control reaction not exposed to UV treatment.

FIG. 5: Effect of the volume on psoralen-based DNA inactivation with a real-time PCR assay based on detection with molecular beacon probes.

Real-time detection on a Smart Cycler using a MRSA-specific PCR assay showing the effect of the volume of the PCR reaction mixture. Purified genomic DNA from Staphylococcus aureus ATCC 33592 (100 genome copies per reaction) was added to all reaction mixtures containing 8-MOP prior to UV treatment. Panel A: DNA inactivation in 0.6 mL plastic tubes of reaction mixture volumes ranging from 100 to 500 μL. Panel B: DNA inactivation in 1.5 mL plastic tubes of reaction mixture volumes ranging from 100 to 1000 μL. The curves (Δ) of each panel correspond to control reactions not exposed to UV treatment.

FIG. 6: Determination of the influence of psoralen-based DNA inactivation with two different concentrations of 8-MOP on the efficiency and analytical sensitivity of a PCR assay.

Melting curves of the PCR products amplified on a LightCycler with the Staphylococcus-specific PCR assay (Tₘ around 83-84° C.) showing the difference on the analytical sensitivity of a PCR assay according to 8-MOP concentration and UV exposure. The peaks in the range of 74 to 82° C. correspond to the Tₘ of non-specific amplification products including primer dimers. Purified genomic DNA from Staphylococcus aureus ATCC 29737 was added after DNA inactivation at concentrations of 2 to 8 genome copies per reaction. Panel A: melting curves after DNA inactivation with 0.06 μg/μL of 8-MOP and UV dose of 2400 mJ/cm², Panel B: DNA inactivation with 0.06 μg/μL of 8-MOP and UV dose of 1500 mJ/cm², Panel C: DNA inactivation with 0.03 μg/μL of 8-MOP and UV dose of 2400 mJ/cm² and Panel D: DNA inactivation with 0.03 μg/μL of 8-MOP and UV dose of 1500 mJ/cm². The curves ( [symbol] ) of each panel correspond to control reactions to which no DNA was added. Curve (Δ) corresponds to 2 genome copies per reaction, curve (∘) corresponds to 4 genome copies per reaction and curve (□) corresponds to 8 genome copies per reaction.

FIG. 7: Efficiency of the psoralen-based DNA inactivation in a real-time PCR assay using molecular beacons.

Real-time detection on a Smart Cycler using a Streptococcus agalactiae-specific PCR assay showing the effect of 8-MOP and UV on a PCR assay using molecular beacons. Purified genomic DNA from S. agalactiae ATCC 12973 was added after DNA inactivation at concentrations of 3 to 100 genome copies per reaction. Panel A: no 8-MOP and no UV exposure, Panel B: Addition of 0.06 μg/μL of 8-MOP and no UV exposure, Panel C: Addition of 0.06 μg/μL of 8-MOP and UV dose of 1500 mJ/cm². The curves ( [symbol] ) of each panel correspond to control reactions to which no DNA was added. Curve (Δ) corresponds to 3 genome copies per reaction, curve (∘) corresponds to 6 genome copies per reaction, curve (□) corresponds to 12 genome copies per reaction, curve (Δ) corresponds to 25 genome copies per reaction, curve (∘) corresponds to 50 genome copies per reaction and curve (□) corresponds to 100 genome copies per reaction.

FIG. 8: Efficiency of psoralen to inactivate TEM DNA contaminating molecular biology grade enzymes.

Conventional PCR amplification with the TEM PCR assay (790-bp amplicon) and the internal control (252-bp amplicon) showing the difference between non treated samples (lanes 1 to 6) and treated with 8-MOP and UV for DNA inactivation (lanes 7 to 12). Lanes 1, 2, 7 and 8 are control reactions to which no DNA sample was added after DNA inactivation. Purified genomic DNA from Eschelichia coli CCRI-9767 carrying the TEM-1 gene was added after DNA inactivation at concentrations of 1 (lanes 3, 4, 9 and 10) and 10 (lanes 5, 6, 11 and 12) genome copies per PCR reaction. A 100-bp molecular size ladder was used (lane M).

FIG. 9: Efficiency of psoralen to inactivate microbial DNA contaminating Taq polymerase preparations.

Melting curves of the PCR products amplified on a LightCycler with a universal PCR assay for bacteria showing the difference between non treated samples and samples treated with 8-MOP and UV for DNA inactivation of microbial DNA naturally present in PCR reagents. Purified genomic DNA from Staphylococcus aureus ATCC 29737 was added after DNA inactivation at concentrations of 10 and 25 genome copies per reaction. The peak at around 83-84° C. corresponds to the specific PCR product amplified from the spiked S. aureus DNA. The peaks in the range of 72 to 82° C. correspond to the Tₘ of non-specific amplification products including primer dimers while those over 86° C. correspond to DNA contamination observed with the untreated reaction mixture. Panel A: Melting curves of untreated samples, Panel B: Melting curves after DNA inactivation with 0.06 μg/μL of 8-MOP and a UV dose of 1500 mJ/cm². The curves ( [symbol] ) of each panel correspond to control reactions to which no DNA was added. Curve (Δ) corresponds to 10 genome copies per reaction and curve (∘) corresponds to 25 genome copies per reaction.

FIG. 10: Influence of the intensity of the UV source on the efficiency of DNA inactivation.

Real-time detection on a Smart Cycler using a MRSA-specific PCR assay showing the effect of UV lamp generating intensities ranging from 1300 to 4200 μW/cm². Purified genomic DNA from S. aureus ATCC 33592 was added after DNA inactivation at concentrations of 1 to 10⁶ genome copies per reaction. Curve (Δ) corresponds to the untreated reactions (i.e. no 8-MOP, no UV treatment). Curve (∘) corresponds to reactions containing 8-MOP but not exposed to UV. Curve (□) corresponds to reactions exposed to a UV source generating an intensity of 4200 μW/cm². Curve (Δ) corresponds to reactions exposed to a UV source generating an intensity of 3700 μW/cm². Curve (□) corresponds to reactions exposed to a UV source generating an intensity of 3200 μW/cm². Curve (⋄) corresponds to reactions exposed to a UV source generating an intensity of 2600 μW/cm². Curve (x) corresponds to reactions exposed to a UV source generating an intensity of 1900 μW/cm². Curve ( [symbol] ) corresponds to reactions exposed to a UV source generating an intensity of 1300 μW/cm².

FIG. 11: Determination of the optimal psoralen concentration for DNA inactivation of PCR reagents.

Real-time detection on a Smart Cycler using a MRSA-specific assay showing fluorescence curves for different 8-MOP concentrations. Purified genomic DNA from S. aureus ATCC 33592 (10⁴ genome copies per reaction) was added to all reaction mixtures prior to DNA inactivation. Panel A: untreated reaction (i.e. no 8-MOP and no UV), Panel B: DNA inactivation with a 8-MOP concentration of 0.015 μg/μL, Panel C: DNA inactivation with a 8-MOP concentration of 0.03 μg/μL, Panel D: DNA inactivation with a 8-MOP concentration of 0.06 μg/μL and Panel E: DNA inactivation with a 8-MOP concentration of 0.12 μg/μL. The curves (Δ) of each panel correspond to control reactions not exposed to UV treatment.

FIG. 12: Determination of the influence of psoralen-based DNA inactivation on the efficiency and analytical sensitivity of a S. agalactiae-specific assay.

Real-time detection on a Smart Cycler using a S. agalactiae-specific assay showing the effect of 8-MOP and UV. Purified genomic DNA from S. agalactiae ATCC 12973 was added after DNA inactivation at concentrations of 2.5 and 5 genome copies per reaction. Panel A: untreated reactions (no 8-MOP and no UV). Panel B: Addition of 0.06 μg/μL of 8-MOP and no UV exposure, Panel C: Addition of 0.06 μg/μL of 8-MOP and UV dose of 1500 mJ/cm². The curves ( [symbol] ) of each panel correspond to control reactions to which no DNA was added. Curves (□) correspond to 2,5 genome copies per reaction and curves (Δ) correspond to 5 genome copies per reaction.

FIG. 13: Determination of the influence of psoralen-based DNA inactivation on the efficiency and analytical sensitivity of a Staphylococcus-specific assay.

Melting curves of the PCR products amplified on a Smart Cycler with the Staphylococcus-specific PCR assay (Tₘ around 83° C.) showing the effect of 8-MOP and UV. Purified genomic DNA from S. aureus ATCC 33592 was added after DNA inactivation at concentrations of 2.5 and 10 genome copies per reaction. Panel A: untreated reactions (no 8-MOP and no UV). Panel B: Addition of 0.06 μg/μL of 8-MOP and no UV exposure. Panel C: Addition of 0.06 μg/μL of 8-MOP and UV dose of 1500 mJ/cm². The curves ( [symbol] ) of each panel correspond to control reactions to which no DNA was added. Curves ( [symbol] ) correspond to 2,5 genome copies per reaction and curves ( [symbol] ) correspond to 10 genome copies per reaction.

The Taq polymerase used in PCR as well as other commercially available enzymes have been shown to be contaminated with bacterial DNA as mentioned above. The use of furocoumarin-based DNA inactivation to prevent amplification of microbial DNA contaminating PCR reagents has been reported (Gale et al., 2003, Clin. Chem. 49:415-424; Corless et al., 2000, J. Clin. Microbiol. 38:1747-1752; Klausegger et al., 1999, J. Clin. Microbiol. 37:464-466; Hughes et al., 1994, J. Clin. Microbiol., 32:2007-2008; Meier et al., 1993, J. Clin. Microbiol. 31:646-652; Jinno et al., 1990, Nucleic Acids Res. 18:6739; and U.S. Pat. No. 5,221,608 and 5,532,145). However, none of the reported methods led to effective and reproducible nucleic acid inactivation without substantial reduction in the performance of the NAT assay mainly because these methods are not properly controlled and not standardized. We demonstrate hereinbelow with many PCR amplification assays that standardization and careful monitoring of the UV treatment is critical to achieve efficient and reproducible psoralen-based nucleic acids inactivation without substantial detrimental effect on the performance of each PCR assay.

The present invention relates to reagents and vessels containing these same reagents for amplification and/or detection of nucleic acids in which the concentration of contaminating nucleic acids is so low, if any, that they do not interfere with the detection of the nucleic acids targeted in the reaction. These reagents include nucleotides and/or nucleotide analogs, oligonucleotides (primers and probes), buffer solution, ions (monovalent and divalent), enzymes (DNA polymerase, RNA polymerase, reverse transcriptase, DNA ligase or any other enzymes used for NAT), amplification facilitators (e.g. betaine, bovine serum albumine, dimethyl sulfoxide, amonium chloride), cryoprotectors (e.g. glycerol), stabilizers (e.g. trehalose) and a solvent (usually water). These reagents containing no or a low level of detectable contaminating DNA may be provided separately, or as separate components of a kit, or mixed together and may be liquid, frozen or dehydrated.

Factors to be monitored are (i) the intensity of the UV source, (ii) the energy dose received by the reagent(s), (iii) the composition of the reagent(s), (iv) the nature of the container and its UV transparency, (v) the volume of the reagent(s), and (vi) the type and the concentration of the furocoumarin compound(s). All these factors should be optimized to inactivate at least 100 copies of spiked control nucleic acids without substantial reduction in the performance of the NAT assay.

Commercially available reagents and kits for the preparation of nucleic acids are often contaminated with bacterial DNA and treatment with DNAase and gamma irradiation are not sufficient to eliminate these nucleic acids (Van der Zee et al., 2002. J. Clin. Microbiol. 40:1126). It is therefore an object of the present invention to provide for cleaner reagents and kits for the preparation of nucleic acids for NAT assays as well as to provide an efficient method to inactivate nucleic acids contaminating said reagents and kits.

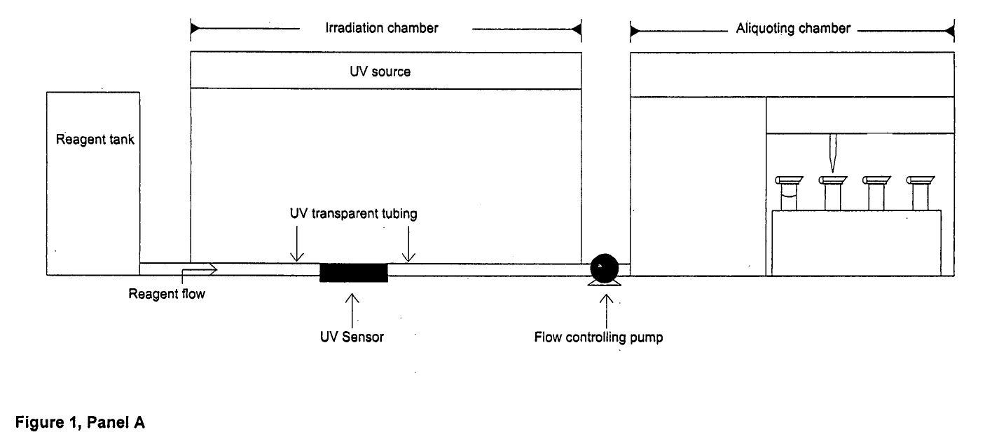

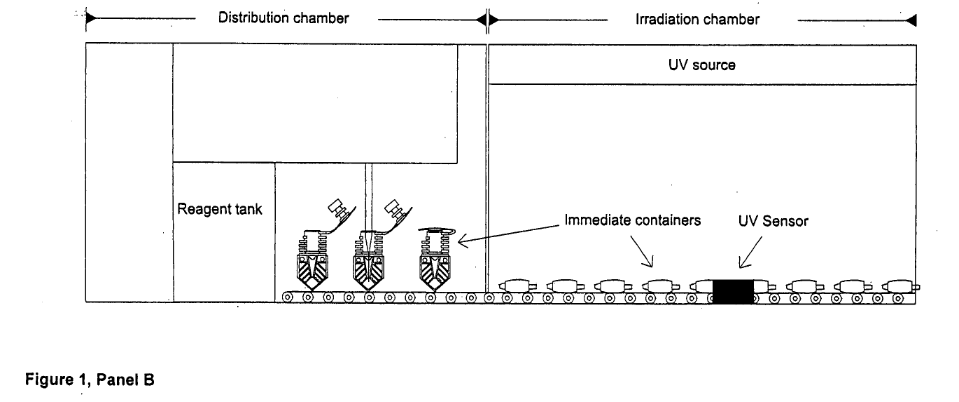

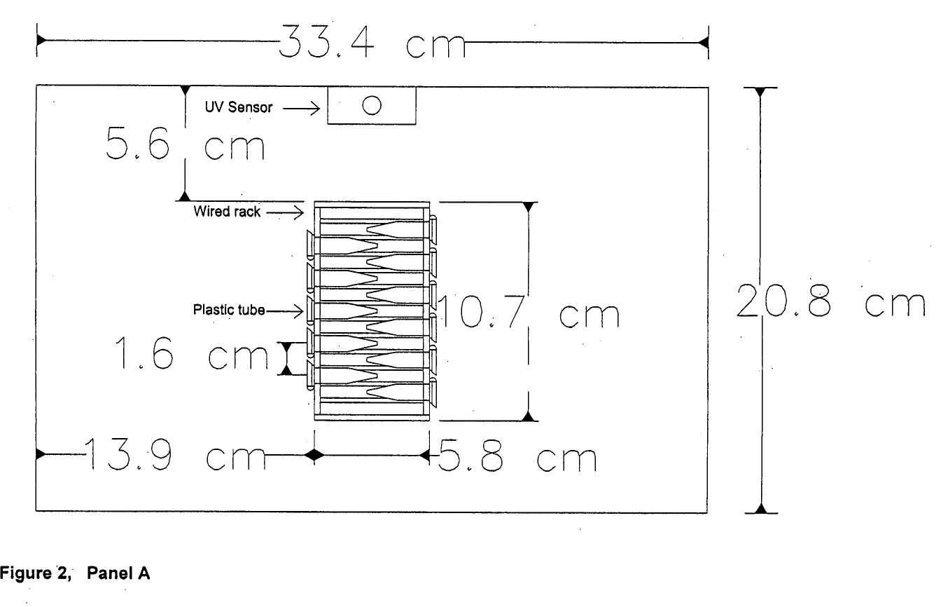

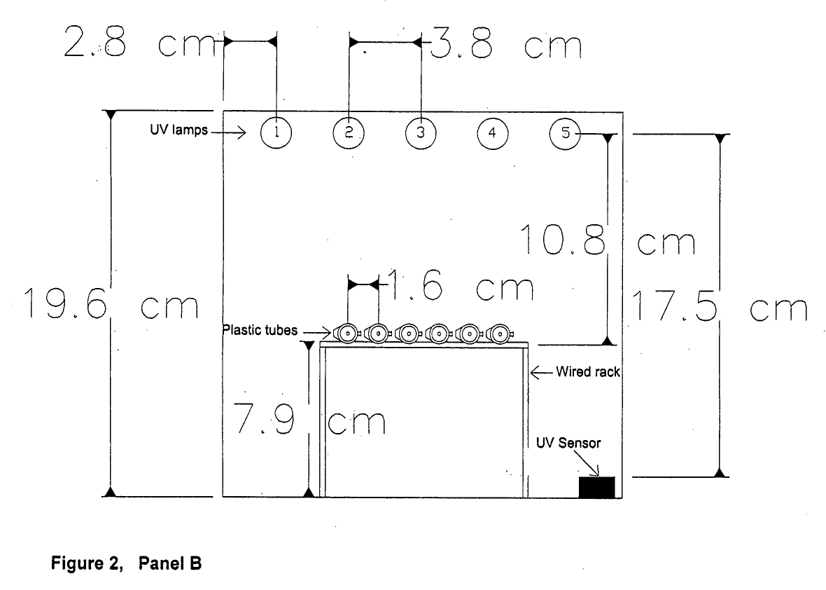

Said nucleic acids amplification and/or detection reagents are preferably treated with an improved method using one or more furocoumarin compound(s) and UV light for nucleic acid inactivation prior to NAT in order to prevent false-positive results, said improved method comprising the following steps.
1) A reaction mixture which contains reagents required for NAT as well as one or more furocoumarin compound(s). The furocoumarin compound used is preferentially a psoralen or isopsoralen derivative. The psoralen derivative is preferentially 8-methoxypsoralen (8-MOP) resuspended in DMSO at a concentration 2.5 mg/mL. The final concentration of 8-MOP in the reaction mixture is of 0.03 to 0.24 μg/μL and preferentially of 0.06 μg/μL (or 0.25 mM). See Example 13 for conditions with nucleic acid inactivation using furocoumarin compounds other than 8-MOP. As mentioned above, a number of sensitive NAT technologies are currently available of which the most widely used is nucleic acid amplification by PCR (see examples). The NAT assay may be performed in liquid phase or onto solid supports. The reaction mixture is preferentially placed into a closed vessel prior to the UV treatment. The closed vessel may be the immediate container in which the NAT is performed or, alternatively, a tubing or a tube. The vessel can be closed, and once closed, evaporation of reagents and/or solvent(s) is avoided. See FIG. 1 for an illustration of a manufacturing process for furocoumarin-based nucleic acid inactivation using a tubing (panel A) or the immediate container (panel B). The closed vessel is preferentially a plastic tube. In a more particular embodiment the vessel is a 0.6 mL plastic tube (such as the MaxyClear flip cap conical tubes from Axygen). On the other hand, the reaction mixture to be treated may have a volume as low as 0.1 mL and as high as 1000 mL depending on the size of the vessel used. The UV treatment is preferentially performed on reaction mixtures placed in vessels which have been validated for furocoumarin-based nucleic acid inactivation because this process is influenced by the quality of the vessel. For example, the composition and thickness of the plastic must be kept constant in order to provide a uniform dosage of UV. Our experience demonstrates that validation for furocoumarin-based nucleic acid inactivation of different lots of vessels from the same manufacturer having identical specifications is important. The reaction mixture volume and the psoralen concentration are also important parameters to optimize.
(2) Said reaction mixture being treated with UV light under controlled conditions wherein the UV exposure as well as the intensity and wavelenght spectrum of the UV source is monitored by using a UV sensor, a radiometer equipped with a UV sensor or a suitable spectrometer. This allowed to ensure that the UV dose was appropriate to inactivate efficiently contaminating nucleic acids without substantial detrimental effect on the performance of the NAT assay. Furthermore, the tight control of the UV treatment was required to achieve an effective and highly reproducible furocoumarin-based nucleic acid inactivation. The reaction mixture would ideally contain all components of the NAT reaction except for the test sample and/or the internal control template to prevent inactivation of target nucleic acids to be detected. The NAT is preferably performed using PCR. The NAT may also be reverse transcriptase PCR (RT-PCR) for RNA detection or any other NAT method. As an example, the following 124 μL PCR reaction mixture can be treated with a controlled UV dose in 0.6 mL plastic tubes. The treated PCR reaction components may include 0.4 μM of each PCR primers, 2.5 mM MgCl₂, 3.3 mg/mL of bovine serum albumin (BSA), 200 μM of each of the four deoxynucleoside triphosphates (dNTPs) (Pharmacia), 10 mM Tris-HCl (pH 9.0), 50 mM KCl, 0.5 X/μL of SYBR Green I (Molecular Probes), 0.5 unit of Taq DNA polymerase (Promega) coupled with TaqStart antibody (Clontech). The test sample is added to each PCR reaction after the UV treatment. If an internal control template is used, it must also be added to the PCR reaction mixture after the standardized UV treatment. Clearly, reagent concentrations other than those mentionned above may be used. Futhermore, other components such as fluorescent probes, detergents or other types of enzymes may also be used. The UV source may be positioned to allow an optimal UV treatment to achieve an efficient furocoumarin-based nucleic acid inactivation of a NAT reaction mixture enclosed into a tube, a tubing or the immediate container (FIG. 1). Alternatively, the UV treatment may be performed by using an apparatus consisting of a chamber equipped with UV lights and a UV sensor to monitor the energy of the treatment in Joule per unit of surface. In a particularly preferred embodiment, said apparatus allowing to monitor the energy of the UV treatment is the Spectrolinker XL-1000 UV Crosslinker (Spectronics Corp.) equipped with UV lamps (wavelenghts spectrum of 320 to 400 nm with an emission peak at around 354 nm based on analysis with a Spectronics SLM-Aminco spectrometer from Thermo Galactic) and a UV sensor. In another particularly preferred embodiment, said reaction mixture is disposed in 0.6 mL plastic containers located at about 10.8 centimeters from the UV source of the Spectrolinker apparatus. FIG. 2 shows the irradiation chamber of the Spectrolinker apparatus. The intensity of the emission peaks of the UV lamps in the UV spectrum may also be monitored by using a radiometer equipped with a UV sensor such as the UVX digital radiometer with a UVX-36 sensor for 365 nm (UVP) or a suitable spectrometer such as the Spectronic SLM-Aminco (Thermo Galactic). See the examples for more specifications on the UV treatment using the Spectrolinker apparatus. Suitable UV sources generating UV light in the wavelength spectrum of 320 to 400 nm include among others a laser, high intensity white light, an incandescent lamp and a diode. The optimal UV treatment is dependent on (i) the distance from the UV source, (ii) the composition and thickness of the used closed reagent vessel or tubing and (iii) the composition and the volume of the NAT reaction mixture.

In order to ascertain the efficiency of the nucleic acid inactivation protocol and the absence of substantial detrimental effect on the performance of the NAT assay, reaction mixtures spiked with the target template as well as reaction mixtures not spiked with the target nucleic acids were used. As shown in the examples, the reaction mixtures were spiked with template nucleic acids targeted by the assay. At least 100 copies of spiked target nucleic acids per PCR reaction containing 0.5 unit of Taq polymerase was preferentially used to evaluate the furocoumarin-based nucleic acid inactivation protocol because it has been demonstrated by our group (data not shown) and others (Rand and Houck, 1990, Mol. Cell. Probes 4:445-450; Meier et al., 1993, J. Clin. Microbiol. 31:646-652) that the most heavily contaminated commercial preparations of Taq polymerase contain approximately 100 to 500 bacterial genomes per unit of enzyme.
3) Said UV treated reaction mixture being subsequently supplemented with the test sample and/or an internal control template. The test sample may be cells, purified nucleic acids or biological specimens preferentially of clinical or environmental source. The target nucleic acid is preferentially microbial DNA. An internal control template nucleic acid targeted by the assay and added to each NAT reaction may be used to verify the efficiency of the reaction and to ensure that there is no significant inhibition by the test sample.
4) Said reaction mixture supplemented with the test sample and/or internal control template is subjected to NAT performed under appropriate conditions. Said nucleic acid amplification technologies include among others, PCR, RT-PCR, LCR, SDA as well as transcription-based amplifications such as TMA. Preferentially, the NAT assay is PCR. The PCR amplification is performed under optimized cycling conditions and amplicon detection can be based (i) on real-time hybridization with internal probes labeled with a fluorophore (e.g. molecular beacons) or, alternatively, (ii) on the incorporation of SYBR Green I and melting curve analysis of the amplification products. Standard agarose gel electrophoresis may also be used for amplicon detection. The examples will provide more details about PCR cycling and real-time or post-amplification amplicons detection. Preferentially, the nucleic acids inactivation process does not have any substantial detrimental effect on the performance of the assay. The performance of the fluorescence-based NAT assays and of the furocoumarin-based nucleic acids inactivation method was monitored by verifying and/or analysing the fluorescence curves, the amplicon melting curves, the analytical sensitivity, the cycle thresholds and/or the fluorescence end points. Standard agarose gel has also been used to verify the performance of the NAT assays and of nucleic acids inactivation.

### EXAMPLES

### EXAMPLE 1

Determination of the Optimal UV Dose for Psoralen-Based DNA Inactivation

The goal of these experiments was to determine the optimal UV exposure to inactivate contaminating DNA in PCR reagents and this without substantial reduction in the performance of the assay.

Method: This evaluation was performed using Staphylococcus-specific PCR primers that we have previously described (Martineau et al., 2001, J. Clin. Microbiol. 39:2541-2547). These primers were used on the Roche LightCycler instrument. PCR amplifications were performed from purified DNA prepared by using the G NOME DNA extraction kit (Bio 101). A master mix containing the equivalent of around 100 S. aureus genome copies per PCR reaction and 0.06 μg/μL of 8-MOP (Sigma) was distributed into 4 aliquots of 124 μL in 0.6 mL plastic tubes (MaxyClear flip cap conical tubes from Axygen). Each aliquot was then treated with UV using a Spectrolinker XL-1000 UV Crosslinker (Spectronics Corp.) equipped with a UV sensor and with UV lamps having a wavelenghts spectrum of 320 to 400 nm with an emission peak at around 354 nm (FIG. 2). The tubes containing the reaction mixture to be treated were placed onto a wire rack support in order to minimize shadowing or obstruction effects on the UV sensor. Up to 11 reaction mixture tubes were placed onto the wire rack positioned in the center of the UV irradiation chamber (FIG. 2) so that the reagent tubes were located at about 10.8 centimeters from the UV source of the Spectrolinker apparatus. The tubes were placed in the middle of the rack when fewer tubes were treated. All tubes were placed on the rack at an angle of 15 to 20 degrees to prevent contact between the reaction mixture to be treated and the tube cap. The intensity of the Spectrolinker five UV lamps could also be measured by using a UVX digital radiometer equipped with a UVX-36 sensor for 365 nm UV (UVP) which was positioned in the middle of the floor of the irradiation chamber. The length of the UV treatment was automatically determined by the apparatus based on the intensity of the UV source as measured by its integrated UV sensor. Aliquots of the PCR master mix were treated with the following UV doses in mJ/cm² measured by the UV sensor of the Spectrolinker apparatus: 1000, 1500, 2000 and 2400 mJ/cm². A total of 7 identical PCR reactions was tested for each UV treated aliquot. Two PCR reactions not treated with UV served as negative controls. Fluorogenic detection of PCR products with the LightCycler was carried out using 0.4 μM of both Staphylococcus-specific PCR primers, 8.0 mM MgCl₂, 0.55 mg/mL of BSA, 200 μM of each of the four dNTPs (Pharmacia), 50 mM Tris-HCl (pH 9.1), 16 mM (NH₄)₂SO₄, 0.5 X/μL of SYBR Green I (Molecular Probes), 1.25 unit of KlenTaq1 (AB Peptides) DNA polymerase coupled with TaqStart antibody (Clontech) and 1 μL of test sample all in a final volume of 15 μL. The KlenTaq1 enzyme is missing the N-terminal portion of the wild-type full length Taq DNA polymerase. The optimal cycling conditions were 1 minute at 94° C. for initial denaturation, and then 45 cycles of three steps consisting of 0 second at 95° C., 5 seconds at 60° C. and 9 seconds at 72° C. Amplification was monitored at each cycle by measuring the level of fluorescence emited by the incorporated SYBR Green I. After the amplification process, melting curves of the amplification products were generated and analysed for each test sample.

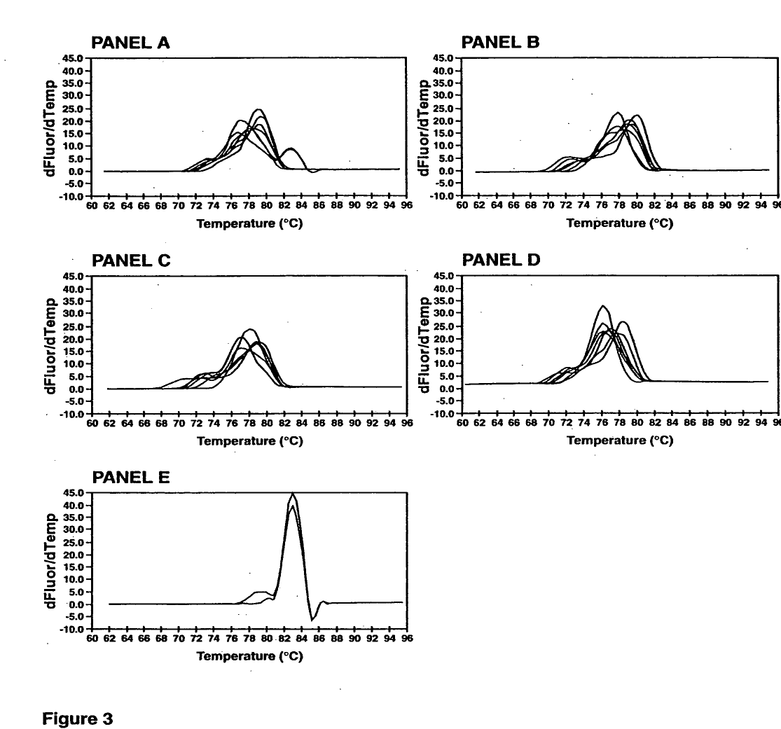

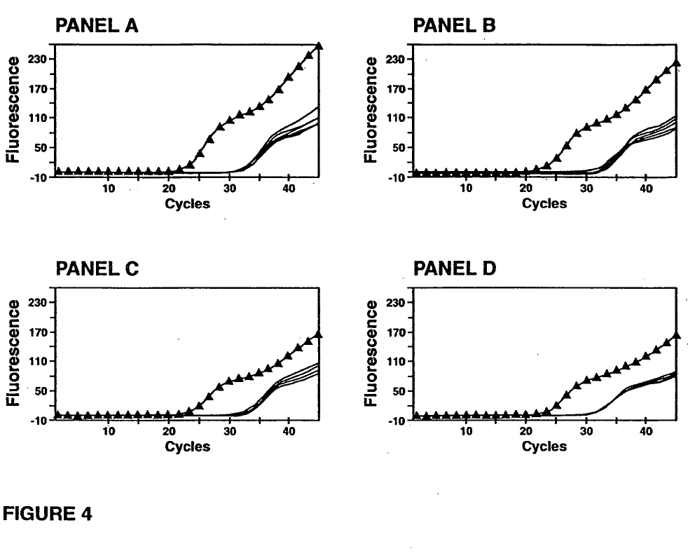

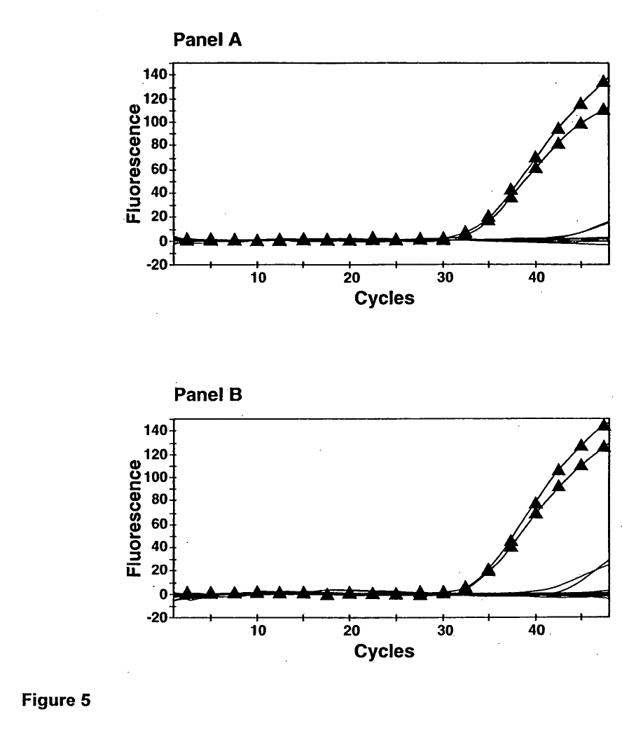

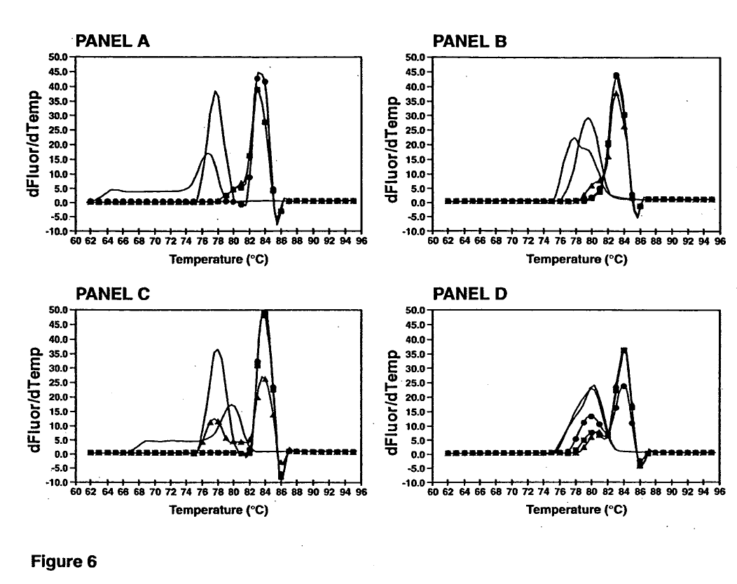

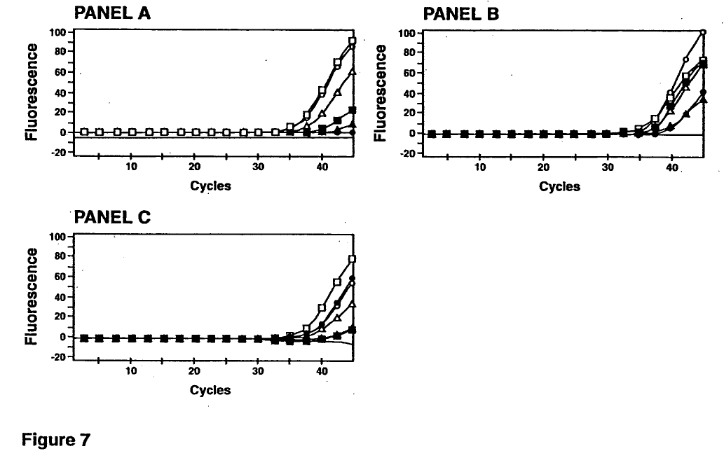

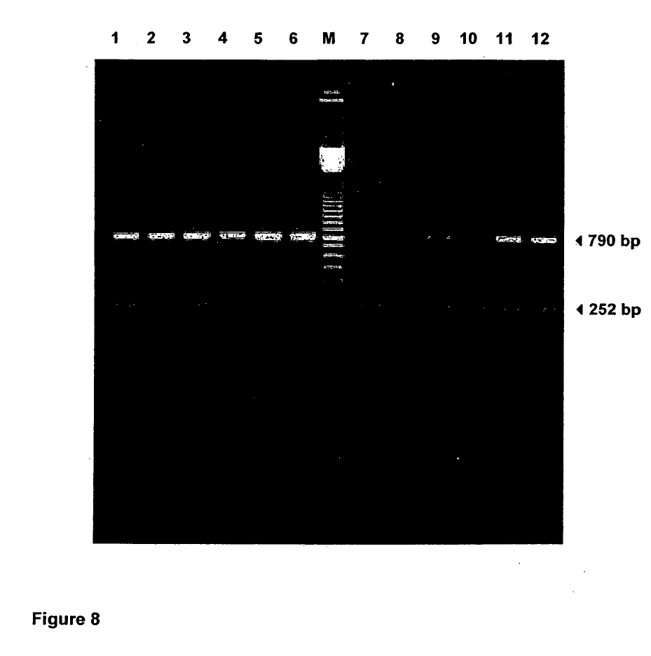

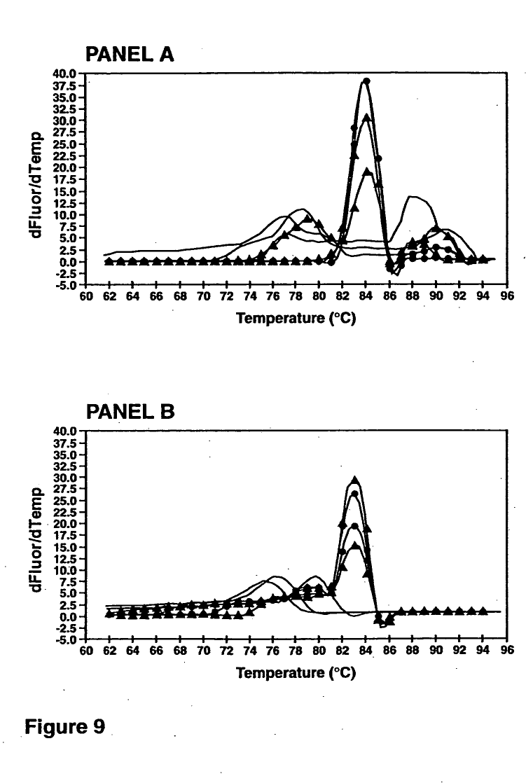

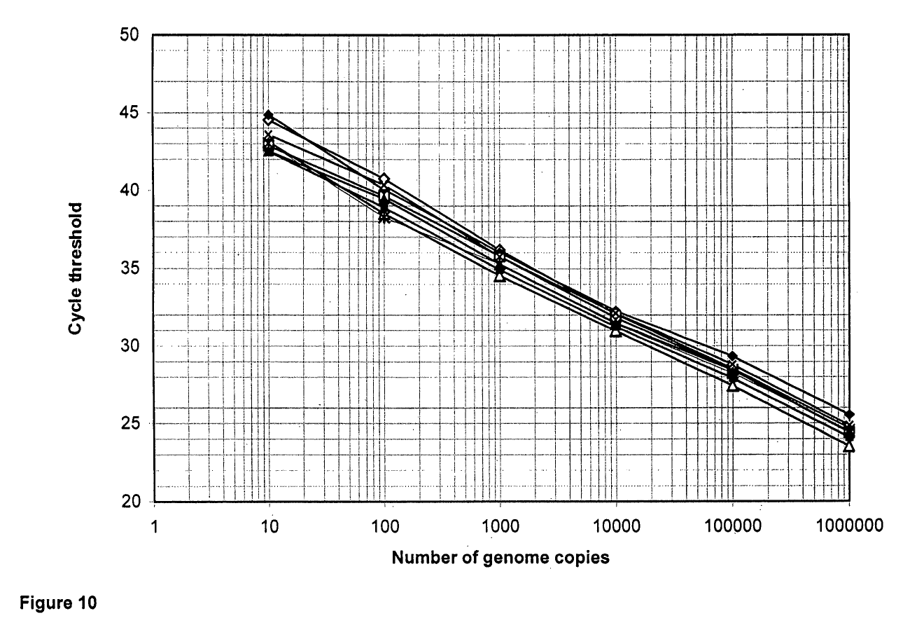

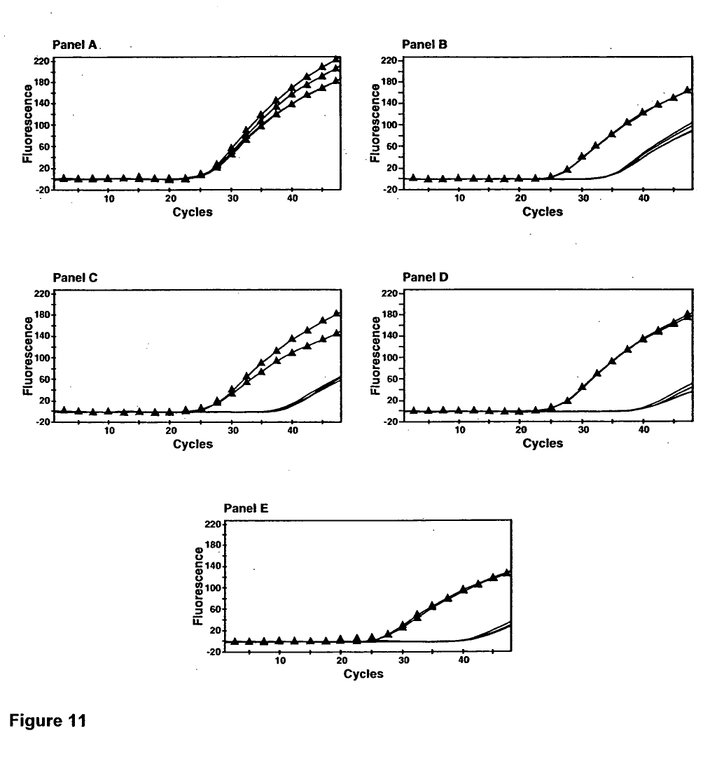

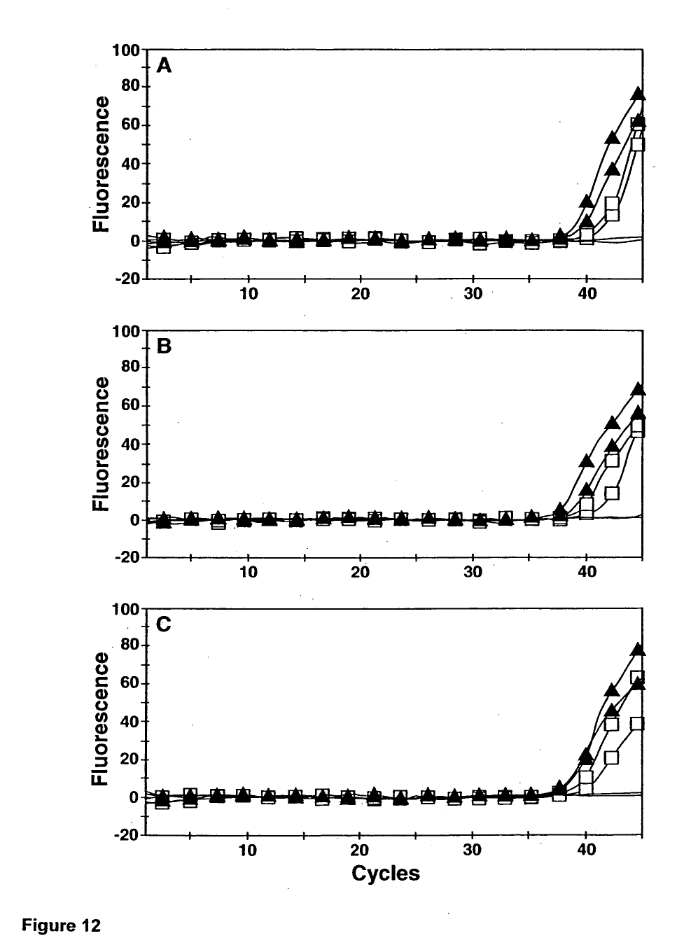

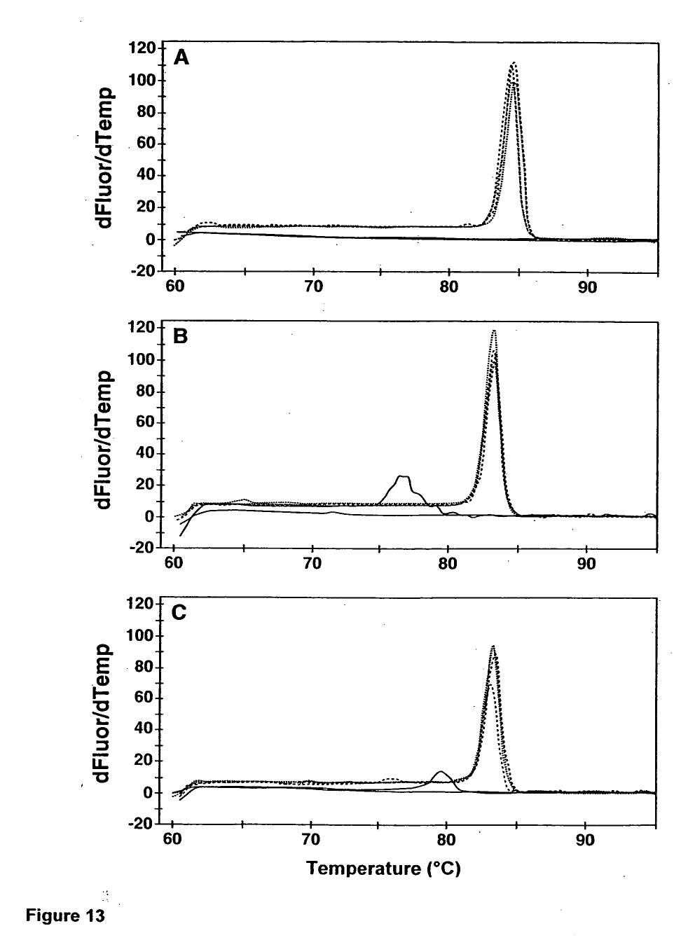

Results and discussion: The inactivation of the spiked S. aureus genomic DNA was complete with UV doses of 1500, 2000 and 2400 mJ/cm² while the inactivation was partial with a dose of 1000 mJ/cm² (FIG. 3). We concluded that, with this system and with these reagents and plastic tubes, the optimal UV dose was 1500 mJ/cm² because it is the lowest effective UV exposure. It should be mentionned that we have also tested a UV dose of 1500 mJ/cm² (measured by the UV sensor of the Spectrolinker apparatus) with other assays amplifying DNA contaminating reagents and found it effective as well (i.e. allowed complete DNA inactivation without substantial detrimental effect on the performance of the assay) (data not shown). Thus, any system capable of providing a UV dose to the treated reagent(s) which is equivalent or comparable to the range of 1500 to 2400 mJ/cm² obtained with the above set-up, system or apparatus is within the scope of this invention.

### EXAMPLE 2

Determination of the Optimal Psoralen Concentration for Decontamination

The objective of these experiments was to determine the optimal psoralen concentration to inactivate DNA in PCR reagents with a S. agalactiae-specific assay.

Method: This evaluation was performed using PCR primers specific for S. agalactiae (also called group B streptococci (GBS)) that we have previously described (Ke et al., 2000, Clin. Chem. 46:324-331). A molecular beacon (FAM-CCACGCCCCAGCAAATGGCTCAAAAGCGCGTGG-DABCYL hybridizing to S. agalactiae-specific amplicons) was synthesized and HPLC-purified by Biosearch Technologies Inc. Purified genomic DNA was prepared as described in Example 1. Amplification reactions were performed using a Smart Cycler thermal cycler (Cepheid) in a 25 μL reaction mixture containing 50 mM Tris-HCl (pH 9.1), 16 mM ammonium sulfate, 8 mM MgCl₂, 0.4 μM of primer Sag59 (5′-TTTCACCAGCTGTATTAGMGTA-3′) and 0.8 μM of primer Sag190 (5′-GTTCCCTGAACATTATCTTTGAT-3′), 0.2 μM of the GBS-specific molecular beacon, 200 μM each of the four dNTPs, 450 μg/mL of BSA, 1.25 unit of KlenTaq1 DNA polymerase (AB Peptides) combined with TaqStart antibody (Clontech), 10⁶ genome copies of S. agalactiae and 0.03 to 0.24 μg/μL of 8-MOP. The 8-MOP concentrations tested for decontaminating the spiked S. agalactiae genomic DNA were 0.03, 0.06, 0.12 and 0.24 μg/μL. For each psoralen concentration, one reaction was not treated with UV while the 7 other reactions were treated with a UV dose of 1500 mJ/cm² (measured by the UV sensor of the Spectrolinker apparatus as described in Example 1). All PCR reaction mixtures were then submitted to thermal cycling (3 min at 94° C., and then 45 cycles of 5 sec at 95° C. for the denaturation step, 14 sec at 56° C. for the annealing step, and 5 sec at 72° C. for the extension step). The GBS-specific amplifications were measured by the increase in fluorescence during the amplification process. Subsequently, 10 μL of each PCR-amplified reaction mixture was also analysed by electrophoresis at 170 V for 30 min, in a 2% agarose gel containing 0.25 μg/mL of ethidium bromide. For agarose gel analysis, the size of the amplification products was estimated by comparison with a 50-bp molecular size standard ladder.

Results and discussion: It was found that the psoralen concentrations of 0.03 μg/μL (0.14 mM) and 0.06 μg/μL (0.28 mM) were the most effective to decontaminate the spiked 10⁶ genome copies of S. agalactiae per PCR reaction (FIG. 4). The cycle thresholds were reduced by about 10 cycles as compared to the control reaction without UV treatment. This corresponds to a decrease of approximately 3 logs in the load of amplifiable S. agalactiae genomic DNA. Regarding, the higher 8-MOP concentrations tested (i.e. 0.12 and 0.24 μg/μL) the fluorescence end points were significantly lower (FIG. 4). The almost perfect overlap of the fluorescence curves for the 7 treated reactions for each psoralen concentration tested demonstrates the excellent reproducibility of this system to inactivate DNA. Importantly, we have tested the 8-MOP concentration of 0.06 μg/μL (0.28 mM) with other PCR assays amplifying DNA contaminating reagents and found it effective as well (i.e. allowed complete DNA inactivation of around 100 spiked genomic bacterial DNA) (data not shown). As far as 8-MOP is concerned a concentration of 0.03 to about 0.09 μg per μL would be preferred, namely about 0.06 μg/μL. Any other compound of the furocoumarin class having the same or comparable potency as this concentration of 8-methoxypsolaren is within the scope of this invention (see Example 13).

### EXAMPLE 3

Determination of the Effect of the Volume on Psoralen-Based DNA Inactivation Using a Staphylococcus-Specific PCR Assay Based on SYBR Green I Detection

The objective of these experiments was to determine if the volume of the reaction mixture had an effect on the efficiency of the process of DNA inactivation by psoralen and UV treatment.

Method: This evaluation was performed using the Staphylococcus-specific PCR assay with purified DNA as described in Example 1. Reaction mixture (containing 0.06 μg/μL of 8-MOP and 100 genome copies of S. aureus per 15 uL of reaction mixture) volumes of 100, 200, 300, 400 and 500 μL were tested in the 0.6 mL plastic tubes described in Example 1. Each reaction volume was treated with a UV dose of 2400 mJ/cm² (measured by the UV sensor of the Spectrolinker apparatus as described in Example 1). Subsequently, each treated volume was used to prepare 6 identical PCR reaction. Two reactions not treated with UV served as negative controls.

Results and discussion: It was found that for all 5 volumes tested, the inactivation of the spiked 100 genome copies of S. aureus was complete based on PCR amplification and detection results (data not shown). We concluded that DNA inactivation for these 5 volumes was effective. Moreover, we have noted that psoralen plus UV treatment is also influenced by the quality of the plastic tubes used. For example, thicker plastic tubes or plastic compounds absorbing more UV may require a stronger UV exposure. We routinely validate each lot of plastic tubes to ensure that they allow efficient psoralen-based DNA inactivation.

### EXAMPLE 4

Effect of the Volume on Psoralen-Based DNA Inactivation with a Real-Time PCR Assay Based Detection with Fluorescent Probes

The objective of these experiments was to determine if the volume of the reaction mixture for a real-time PCR assay had an effect on the efficiency of the process of DNA inactivation by psoralen and UV treatment.

Method: This evaluation was performed using a PCR assay for the specific detection of methicillin-resistant Staphylococcus aureus (MRSA). PCR amplifications were performed from purified DNA as described in Example 1. Reaction mixture (containing 0.06 μg/μL of 8-MOP and 100 genome copies of a MRSA strain per 15 uL of reaction mixture) volumes of 100, 200, 300, 400 and 500 μL were treated in the 0.6 mL plastic tubes described in Example 1. Also, volumes of 100, 200, 500 and 1000 μL of the same PCR reaction mixture containing 8-MOP and spiked MRSA genomic DNA were treated in 1,5 mL plastic tubes tubes (MaxyClear flip cap conical tubes from Axygen). Each reaction volume was treated with a UV dose of 1500 mJ/cm² (measured by the UV sensor of the Spectrolinker apparatus as described in Example 1). Subsequently, each treated volume was used to prepare 4 identical PCR reactions. Two reactions not treated with UV served as negative controls. Amplification reactions were performed using a Smart Cycler thermal cycler (Cepheid) in a 25 μL reaction mixture containing 100 genome copies of an MRSA strain added prior to the UV treatment, 0.8 μM of XSau325 primer (5′-GGATCMACGGCCTGCACA-3′), 0.4 μM of mec1V511 primer (5′-CAAATATTATCTCGTAATACCTTGTTC-3′), 0.2 μM of XSau-B5-A0 molecular beacon (FAM-CCCGCGCGTAGTTACTGCGTTGTMGACGTCCGCGGG-DABCYL), 3.45 mM MgCl₂, 3.4 mg/mL of BSA, 330 μM of each of the four dNTPs, 10 mM Tris-HCl (pH 9.0), 50 mM KCl, 0.035 unit of Taq DNA polymerase (Promega) coupled with TaqStart antibody and 1 μL of test sample. The optimal cycling conditions were 3 minute at 95° C. for initial denaturation, and then 48 cycles of three steps consisting of 5 second at 95° C., 15 seconds at 60° C. and 15 seconds at 72° C. The MRSA-specific amplifications were measured by the increase in fluorescence during the amplification process. Subsequently, 10 μL of each PCR-amplified reaction mixture was also analysed by electrophoresis as described in Example 2.

Results and discussion: It was found that for all 5 volumes tested in 0.6 mL tubes, the inactivation of the spiked genomic DNA of S. aureus was complete most of the times based on PCR amplification and detection results (FIG. 5). One out of the four replicates for the tubes containing 200 μL or 400 μL of treated reaction mixture showed an almost complete DNA inactivation. Regarding inactivation in 1.5 mL tubes, all four replicates showed complete inactivation of the spiked genomic DNA for the tested volumes of 100, 200 and 500 μL (FIG. 5). Two out of the four reactions in the tubes containing 1000 μL showed an almost complete DNA inactivation. This may be associated with an insufficient UV exposure for this larger volume. Comparison with a reaction mixture containing 8-MOP and not treated with UV revealed that there was no substantial reduction in the performance of the assay associated with the nucleic acid inactivation method (data not shown). These results demonstrate the versatility of this method to inactivate nucleic acids found in various volumes of PCR reaction mixtures enclosed in different types of plastic containers.

### EXAMPLE 5

Influence of Psoralen-Based DNA Inactivation with Two Different Concentrations of 8-MOP on the Analytical Sensitivity of a PCR Assay

The objective of these experiments was to determine if DNA inactivation by using two different concentrations of 8-MOP and UV treatment has an influence on the efficiency of the process of DNA amplification by PCR.

Method: This evaluation was performed using the Staphylococcus-specific PCR assay with purified DNA as described in Example 1. A volume of 132 μL containing no S. aureus DNA and 0.03 or 0.06 μg/μL of 8-MOP was treated with an energy dose of 1500 or 2400 mJ/cm² (measured by the UV sensor of the Spectrolinker apparatus as described in Example 1). Sensitivity assays were performed by adding to 15 μL aliquots two-fold dilutions of purified S. aureus genomic DNA after the UV treatment. The numbers of genome copies per PCR reaction tested were 2, 4, 8, 16 and 32. There was 2 negative control reactions to which no S. aureus DNA was added. The performance of the assay was monitored by verifying the analytical sensitivity of the assay based on amplicon melting curves analysis. Analysis of the fluorescence curves and of the amplicon melting curves was also performed.

Results and discussion: It was demonstrated that there was no substantial decrease in the analytical sensitivity of the PCR assay with reaction mixtures submitted to the four different psoralen treatments (FIG. 6) as compared with an untreated reaction mixture (i.e. no 8-MOP and no UV). Analysis of the fluorescence curves and of the amplicon melting curves did not revealed any substantial difference in the performance of the assay for the two 8-MOP concentrations and two UV doses tested. In conclusion, this optimized method for psoralen-based DNA inactivation does not interfere significantly with the PCR assay. This is crucial because apparent DNA inactivation may in fact be attributable to a reduction in the performance of the assay.

### EXAMPLE 6

Efficiency of the Psoralen-Based DNA Inactivation with Different Polymerases

The objective of these experiments was to determine if DNA inactivation by psoralen and UV treatment has an influence on the efficiency of the Taq and KlenTaq1 polymerases.

Method: This evaluation was performed using the Staphylococcus-specific PCR assay. The performance of this assay using either the Taq polymerase from Roche (as described in Example 9 except that the universal primers were not used) or the KlenTaq1 polymerase from AB Peptides (as described in Example 1) was compared. Both enzymes were coupled with the TaqStart antibody. The concentration of Taq polymerase was 0.025 unit/μL while that of KlenTaq1 was 0.125 unit/μL. A volume of 132 μL containing no S. aureus DNA and 0.06 μg/μL of 8-MOP was treated with UV lamps generating an energy of 1500 or 2400 mJ/cm² (measured by the UV sensor of the Spectrolinker apparatus as described in Example 1). Sensitivity assays were performed by adding two-fold dilutions of purified S. aureus genomic DNA to 15 μL aliquots of the treated PCR reaction mixtures. The numbers of genome copies per PCR reaction tested were 2, 4, 8, 16 and 32. There was two negative control reactions to which no S. aureus DNA was added.

Results and discussion: It was demonstrated that there was no subtantial decrease in the analytical sensitivity of the PCR assay with reaction mixtures submitted to the two different psoralen treatments (i.e. (i) 0.06 μg/μL of 8-MOP with a UV dose of 1500 mJ/cm² and (ii) 0.06 μg/μL of 8-MOP with a UV dose of 2400 mJ/cm²) as compared with an untreated reaction mixture (i.e. no 8-MOP and no UV treatment) (data not shown). Therefore, our optimized method for psoralen-based DNA inactivation does not interfere substantially with the PCR assay using these two polymerases. This is crucial because apparent DNA inactivation may in fact be attributable to a reduction in the performance of the assay.

### EXAMPLE 7

Efficiency of the Psoralen-Based DNA Inactivation in a Real-Time PCR Assay Using Fluorescent Probes

The objective of these experiments was to determine if DNA inactivation by psoralen and UV treatment has an influence on the efficiency of a real-time PCR assay using fluorescent probes.

Method: This evaluation was performed using the PCR assay specific for S. agalactiae described in Example 2 except that an additional molecular beacon (TET-CCACGCGAAAGGTGGAGCAATGTGMGGCGTGG-DABCYL) targeting the internal control template was used. The internal control was used to verify the efficiency of the PCR and to ensure that there was no significant PCR inhibition by the test sample. A volume of 132 μL of PCR reaction mixture containing no S. agalactiae DNA and 0.06 μg/μL of 8-MOP was treated with a UV dose of 1500 mJ/cm² (measured by the UV sensor of the Spectrolinker apparatus as described in Example 1). After the UV treatment, the equivalent of 100 copies per PCR reaction of the internal control template were added. Sensitivity assays were performed by adding two-fold dilutions of purified S. agalactiae genomic DNA to 15 μL aliquots of the treated PCR reaction mixture. The numbers of genome copies per PCR reaction tested were 3, 6, 12, 25, 50 and 100. There was 2 negative control reactions to which no S. agalactiae DNA was added. The performance of the assay was monitored by verifying three parameters including the analytical sensitivity of the assay, the cycle thresholds and the fluorescence end points.

Results and discussion: There was no substantial decrease in the performance of the PCR assay associated with the psoralen-based DNA inactivation as revealed by a comparison with the untreated reaction mixtures (FIG. 7). The internal control template was amplified normally for all PCR reactions thereby confirming the efficiency of each PCR amplification and detection using the molecular beacon specific to the internal control (data not shown).

### EXAMPLE 8

Efficiency of Psoralen to Inactivate TEM DNA Contaminating Molecular Biology Grade Enzymes

The objective of these experiments was to determine if DNA inactivation by psoralen and UV treatment is effective to inactivate TEM DNA (coding for a beta-lactamase) which is frequently found in enzyme and other reagent preparations.

Method: This evaluation was performed using a PCR assay specific for the beta-lactamase gene TEM described in our co-pending patent application WO 0123604 A (SEQ ID Nos. 1907 and 1908). Internal control primers and template were used as previously described (Lansac et al., 2000, Eur. J. Clin. Microbiol. Infect. Dis. 19:443-451). The internal control was used to verify the efficiency of the PCR and to ensure that there was no significant PCR inhibition by the test sample. Standard PCR amplifications were carried out on a PTC-200 thermocycler (MJ Research) using purified DNA prepared as described in Example 1. The PCR reaction mixture contained 0.06 μg/μL of 8-MOP, 50 mM KCl, 10 mM Tris-HCl (pH 9.0), 0.1% Triton X-100, 2.5 mM MgCl₂, 0.4 μM (each) of the TEM-specific primers, 200 μM (each) of the four dNTPs, 3.3 mg/mL of BSA and 0.5 unit of Taq polymerase (Promega) coupled with TaqStart antibody and 1 μL of test sample all in a final volume of 20 μL. This reaction mixture was treated with a UV exposure of 1500 mJ/cm² (measured by the UV sensor of the Spectrolinker apparatus as described in Example 1). Another identical reaction mixture without the 8-MOP and not treated with UV was also tested. The equivalent of 1 or 10 genome copies of Escherichia coli strain CCRI-9767 (strain RbK, TEM-1) carrying a TEM plasmid were added to two reactions after the UV treatment. The optimal cycling conditions were 3 minute at 94° C. for initial denaturation, and then 35 cycles of three steps consisting of 5 seconds at 95° C., 30 seconds at 55° C. and 30 seconds at 72° C. followed by a terminal extension of 5 minutes at 72° C. Detection of the PCR products was performed by electrophoresis as described in Example 2.

Results and discussion: It was demonstrated that the psoralen plus UV treatment allowed complete inactivation of the TEM DNA present in Taq DNA polymerase preparations (FIG. 8). The internal control was amplified normally for all PCR reactions thereby confirming their efficiency. We have tested many lots of Taq polymerase from various manufacturers and found that all of them were contaminated with TEM DNA. Surprisingly, this TEM contamination is also observed with native Taq polymerase purified from Thermus aquaticus. The source of TEM DNA is likely cloning vectors manipulated in the laboratories where the proteins are purified or in the laboratories in which NAT is performed. In conclusion, this psoralen-based DNA inactivation method performed prior to TEM DNA amplification and detection is effective to avoid false-positive results.

### EXAMPLE 9

Efficiency of Psoralen to Inactivate Microbial DNA Contaminating Taq Polymerase Preparations

The objective of these experiments was to determine if DNA inactivation by the improved psoralen and UV treatment is effective to inactivate microbial DNA contaminating Taq DNA polymerase preparations in order to prevent false-positive results with a universal PCR assay for bacteria.

Method: This evaluation was performed using a multiplex PCR assay targeting the tuf gene for the universal detection of bacteria. This PCR assay included universal primers that we have previously described (SEQ ID Nos 636 and 637 of our co-pending patent application PCT/CA00/01150) as well as the Staphylococcus-specific PCR primers (Martineau et al., 2001, J. Clin. Microbiol. 39:2541-2547). Amplification reactions were performed using the Roche LightCycler plafform with purified DNA as described in Example 1. Each 15 μL reaction mixture contained 0.4 μM of both Staphylococcus-specific PCR primers, 1.0 μM of both universal primers, 2.5 mM MgCl₂, 2.0 mg/mL of BSA, 200 μM of each of the four dNTPs, 10 mM Tris-HCl (pH 8.3), 50 mM KCl, 0.5 X/μL of SYBR Green I, 0.5 unit of Taq DNA polymerase (Roche) coupled with TaqStart antibody, 0.06 μg/μL of 8-MOP and 1 μL of test sample. This reaction mixture was treated with a UV exposure of 1500 mJ/cm² (measured by the UV sensor of the Spectrolinker apparatus as described in Example 1). Another identical reaction mixture without 8-MOP and not treated with UV was also tested. For each mixture, there was 2 positive control reactions to which the equivalent of 10 genome copies of S. aureus strain ATCC 29737 were added after the UV treatment. Two other positive control reactions to which the equivalent of 25 genome copies of S. aureus were added after the UV treatment were also used. The optimal cycling conditions were 1 minute at 94° C. for initial denaturation, and then 45 cycles of three steps consisting of 0 second at 95° C., 10 seconds at 60° C. and 20 seconds at 72° C. Amplification products analysis was performed as described in Example 1.

Results and discussion: It was demonstrated that the psoralen plus UV treatment allowed complete inactivation of bacterial genomic DNA contaminating Taq DNA polymerase preparations and this without substantial detrimental effect on the performance of the PCR assay (FIG. 9). On the other hand, the untreated PCR reaction mixture led to false-positive results. We have noted lot to lot variations in the load of contaminating DNA in Taq polymerase preparations and found that the most heavily contaminated preparations contained a maximum of about 100 microbial genome copies per unit of enzyme. In conclusion, this psoralen-based DNA inactivation method performed prior to universal (or broad-range) amplification and detection is effective to avoid false-positive results.

### EXAMPLE 10

Examples of Automated Systems for Manufacturing Processes Allowing Controlled UV Treatments and Aliquoting of the Treated Reagents.

The process for furocoumarin-based nucleic acids inactivation may be automated for large-scale production. FIG. 1 illustrates examples of automated systems for manufacturing processes using either a tubing (panel A) or the immediate container (panel B). These systems allow a controlled UV treatment and aliquoting of the treated reagents.

The system using a UV transparent tubing is equipped with a pump allowing to control the flow of the NAT reaction mixture in such a way that the exposition to the controlled UV source is optimal for nucleic acid inactivation without substantial detrimental effect on the NAT reagents. The reagents are subsequently aliquoted in the NAT reaction vessels. The test sample and/or the internal control template are then added to each vessel.

The system using the immediate container automates aliquoting in these vessels as well as the appropriate exposure to the UV source in order to achieve optimal nucleic acid inactivation without substantial detrimental effect on the NAT reagents.

### EXAMPLE 11

Determination of the Optimal UV Dose for Psoralen-Based DNA Inactivation

The goal of these experiments was to determine the optimal UV energy dose to inactivate contaminating DNA in PCR reagents and this without substantial reduction in the performance of the assay.

Method: This evaluation was performed using the MRSA-specific assay described in Example 4. Purified genomic DNA was prepared as described in Example 1. Amplifications were performed using a Smart Cycler in a 25 μL reaction mixture containing 10⁵ genome copies of S. aureus added prior to UV treatment. The 8-MOP concentration used to inactivate the spiked S. aureus genomic DNA was 0.06 μg/μL. The UV energy doses tested were 750, 1500, 3000, 4500 and 6000 mJ/cm². Inactivation treatments were achieved in 0.6 mL plastic tubes described in Example 1. Two reactions were not treated with UV while 6 reactions were treated for each of the UV doses tested (measured by the UV sensor of the Spectrolinker apparatus as described in Example 1). The performance of the MRSA-specific assay was verified for each UV exposure as follows and compared to untreated reaction (no 8-mop and no UV). A volume of 224 μL containing no S. aureus DNA and 0.06 μg/μL of 8-MOP was treated with an energy dose of 750 to 6000 mJ/cm². Sensitivity assays were performed in duplicate by adding different amounts of purified S. aureus genomic DNA to 25.5 μL aliquots of each treated PCR reaction mixture. The numbers of genome copies per PCR reaction tested were 2.5, 5 and 10. There were 2 negative control reactions to which no S. aureus DNA was added. All PCR reaction mixtures were then submitted to thermal cycling as described in Example 4. The performance of the assay was monitored by verifying two parameters including the analytical sensitivity of the assay and the cycle thresholds.

Results and discussion: All UV exposures tested allowed efficient inactivation of the spiked 10⁵ genome copies of S. aureus per PCR reaction (Table 5). The DNA inactivation using the different UV exposures led to an increase in cycle thresholds ranging from about 7 cycles for the UV treatment of 750 mJ/cm² to about 18 cycles for the UV tyreatment of 6000 mJ/cm² as compared to the control reactions containing no 8-MOP and not exposed to UV (Table 5). This corresponds to a decrease of approximately 2 to 4 logs in the load of amplifiable S. aureus genomic DNA. Again, the almost perfect overlap of the fluorescence curves for the six treated reactions for each UV energy dose tested demonstrates the excellent reproducibility of this system to inactivate DNA (data not shown).

There was no substantial variation in the analytical sensitivity as well as in the cycle thresholds with reaction mixtures submitted to a UV dose of 750, 1500 or 3000 mJ/cm² as compared with an untreated reaction mixture (i.e. no 8-MOP and no UV) (Table 5). On the other hand, there was a more important variation in the analytical sensitivity and/or in the cycle thresholds with reaction mixtures submitted to a UV dose of 4500 or 6000 mJ/cm² as compared with the untreated reaction mixture (i.e. no 8-MOP and no UV) (Table 5). The cycle tresholds were increased by about 5 cycles for the UV dose of 4500 mJ/cm² but the assay still allowed the detection of 2.5 genome copies with this UV treatment. For a UV dose of 6000 mJ/cm², there was an important reduction of the analytical sensitivity as revealed by the inability to detect 2.5 and 5 genome copies. Therefore, the apparent increase in DNA inactivation activity of a reaction mixture exposed to the UV doses of 4500 and 6000 mJ/cm² was partly attributable to a detrimental effect on the PCR reagents. In conclusion, this optimized method for psoralen-based DNA inactivation did not reduce substantially the performance of the PCR assay with UV exposures ranging from 750 to 4500 mJ/cm².

### EXAMPLE 12

Influence of the Intensity of the UV Source on the Efficiency of DNA Inactivation

Method: This evaluation was performed using the MRSA-specific assay described in Example 4. PCR amplifications were performed from purified DNA as described in Example 1. Amplifications were performed using a Smart Cycler thermal cycler (Cepheid) in a 25 μL reaction mixture containing 10⁵ genome copies of S. aureus added prior to UV treatment. The 8-MOP concentration was 0.06 μg per μL of reaction mixture. Nucleic acid inactivation treatment was achieved in 0.6 mL plastic tubes described in Example 1. For each UV source intensities tested, two reactions were not treated with UV while 6 other reactions were treated with a UV dose of 1500 mJ/cm² using a UV source generating intensities ranging from 1300 to 4200 μW/cm² (measured by the UV sensor of the Spectrolinker apparatus as described in Example 1). Intensities of 4200, 3700 and 3200 μW/cm² were generated by the five UV lamps of the apparatus. Lower intensities were generated using fewer lamps because the lamp intensities could not be reduced further even after prolonged usage: (i) intensities of 2600 were generated using 4 lamps (# 2 to 5); (ii) intensities of 1900 were generated using 3 lamps (# 3 to 5); and (iii) intensities of 1300 were generated using 2 lamps (# 4 and 5) (FIG. 2). The performance of the MRSA-specific assay was verified for each set of lamps as follows. A volume of 224 μL containing no S. aureus DNA and 0.06 μg/μL of 8-MOP was treated with the UV lamps generating an intensity in the range of 1300 to 4200 μW/cm² and an energy dose of 1500 mJ/cm² (both measured by the UV sensor of the Spectrolinker apparatus). The UV source intensities tested were 4200, 3700, 3200, 2600, 1900 and 1300 μW/cm². Sensitivity assays were performed by adding ten-fold dilutions of purified S. aureus genomic DNA to 25.5 μL aliquots of the treated PCR reaction mixture. The numbers of genome copies per PCR reaction tested were 1, 10, 10², 10³, 10⁴, 10⁵ and 10⁶ There was 2 negative control reactions to which no S. aureus DNA was added. All PCR reaction mixtures were then submitted to thermal cycling as described in Example 4. The performance of the assay was monitored by verifying two parameters including the analytical sensitivity of the assay and the cycle thresholds. Results and discussion: All UV source intensities tested allowed similar efficiencies of inactivation of the spiked 10⁵ genome copies of S. aureus per PCR reaction (Table 1). The DNA inactivation using the different UV source intensities led to an increase in the cycle thresholds of about 10 to 13 cycles as compared to the control reactions containing 8-MOP but not exposed to UV treatment (Table 1). This corresponds to a decrease of approximately 3 logs in the load of amplifiable S. aureus genomic DNA. Again, the almost perfect overlap of the fluorescence curves for the six treated reactions for each UV source intensity tested demonstrates the excellent reproducibility of this system to inactivate DNA (data not shown).

There was no substantial variation in the analytical sensitivity as well as in the cycle thresholds with reaction mixtures submitted to the different UV lamp intensities as compared with an untreated reaction mixture (i.e. no 8-MOP and no UV) (FIG. 10). Therefore, this optimized method for psoralen-based DNA inactivation did not interfere significantly with the PCR assay in the range of UV source intensities tested with the same UV dose.

### EXAMPLE 13

Use of Different Furocoumarin Compounds for Nucleic Acid Inactivation

Method: We have tested furocoumarin compounds other than 8-MOP including psoralen, angelicin, 4-aminomethyltrioxalen, trioxalen, HQ (1,4,6,8-tetramethyl-2H-furo[2,3-h]quinolin-2-one) and HFQ (4,6,8,9-tetramethyl-2H-furo[2,3-h]quinolin-2-one), using the MRSA-specific PCR assay described in Example 4. For each furocoumarin, a range of concentrations and of UV doses were tested in order to determine the optimal conditions for effective DNA inactivation without (if possible) substantial detrimental effect on the performance of the assay (Table 2).

Results and discussion: The optimal concentration for each furocoumarin tested was initially determined by verifying the performance of the PCR assay in the presence of different concentrations of each compound in the absence of UV treatment. The optimal concentration varied widely depending on the furocoumarin (i.e. ranged from 0.003 to 0.06 μg/μL) (Table 2). This was attributable to the highly variable detrimental effect of these furocoumarins (without UV treatment) on the performance of the assay. For example, concentrations of trioxsalen higher than 0.003 μg/μL inhibited partially or completely the PCR assay as opposed to 8-MOP which was found to be optimal at around 0.06 μg/μL. Subsequently, the optimal UV dose in the range of 320 to 400 nm using the preestablished optimal concentration for each furocoumarin was determined. The optimal UV dose also varied depending on the furocoumarin (i.e. ranged from 500 to 1500 mJ/cm² as measured by the UV sensor of the Spectrolinker apparatus as described in Example 1) (Table 2).

The use of each compound in their respective optimal conditions revealed that the only furocoumarin not reducing the analytical sensitivity of the assay and allowing efficient DNA inactivation were 8-MOP and trioxsalen. Trioxalen was shown to be effective at concentrations in the range of 0.001 μg/μL (0.0044 mM) to 0.0075 μg/lμL (0.033 mM) with UV doses ranging from 500 to 1500 mJ/cm². Psoralen and FQ reduced the sensitivity of the assay by about 1 log and 2 logs, respectively (Table 2). Angelicin, 4-aminomethyltrioxsalen and HFQ reduce by more than two-logs the analytical sensitivity of the PCR assay. It was concluded that the best furocoumarins for effective DNA inactivation without substantial detrimental effects on the performance of the assay were 8-MOP and trioxsalen.

### EXAMPLE 14

Determination of the Optimal Psoralen Concentration for DNA Inactivation of PCR Reagents

The objective of these experiments was to determine the optimal psoralen concentration to inactivate DNA in PCR reagents with a MRSA-specific assay.

Method: This evaluation was performed using the MRSA-specific assay described in Example 4. Purified genomic DNA was prepared as described in Example 1. Amplifications were performed using a Smart Cycler in a 25 μL reaction mixture containing 104 genome copies of S. aureus added prior to UV treatment. The 8-MOP concentrations tested to inactivate the spiked S. aureus genomic DNA were 0.015, 0.03, 0.06 and 0.12 μg per μL of reaction mixture. Inactivation treatments were achieved in 0.6 mL plastic tubes described in Example 1. For each psoralen concentration, two reactions were not treated with UV while 4 reactions were treated with a UV dose of 1500 mJ/cm² (measured by the UV sensor of the Spectrolinker apparatus as described in Example 1). The performance of the MRSA-specific assay was verified for each 8-MOP concentration as follows. A volume of 224 μL containing no S. aureus DNA and 0.015, 0.03, 0.06 or 0.12 μg/μL of 8-MOP was treated with an energy of 1500 mJ/cm². Sensitivity assays were performed by adding different amounts of purified S. aureus genomic DNA to 25.5 μL aliquots of each treated PCR reaction mixture. The numbers of genome copies per PCR reaction tested were 2.5, 5 and 10. There were 2 negative control reactions to which no S. aureus DNA was added. All PCR reaction mixtures were then submitted to thermal cycling as described in Example 4. The performance of the assay was monitored by verifying two parameters including the analytical sensitivity of the assay and the cycle thresholds.

Results and discussion: Cycle thresholds observed with the untreated reaction containing 0.015 to 0.12 μg/μL of 8-MOP were similar to the untreated reactions containing no 8-MOP (FIG. 11, panel A). The fluorescence end points for the untreated reaction containing 0.015 to 0.12 μg/μL of 8-MOP were also comparable to the untreated reactions containing no 8-MOP. The most important reduction in the fluorescence end points (30 to 40% decrease) was observed with the highest psoralen concentration tested. These results demonstrate that all 8-MOP concentrations tested did not have a substantial detrimental effect on the performance of the assay.

The cycle thresholds observed with the four different concentrations of 8-MOP exposed to UV treatment were increased by about 10 to 15 cycles as compared to control reactions not exposed to UV (FIG. 11). This corresponds to a decrease of approximately 3 to 4 logs in the load of amplifiable S. aureus genomic DNA. Again, the almost perfect overlap of the fluorescence curves for the four treated reactions for each psoralen concentration tested demonstrates the excellent reproducibility of this system to inactivate DNA. The reaction mixtures submitted to UV treatment in the presence of the four different concentrations of 8-MOP showed no substantial decrease in terms of analytical sensitivity and cycle thresholds as compared with an untreated reaction mixture (i.e. no 8-MOP and no UV) (data not shown). The highest 8-MOP concentration tested (i.e. 0.12 μg/μL) showed a more important increase in the cycle thresholds but the assay still allowed the detection of 2.5 copies of S. aureus genome per PCR reaction (Table 3). Therefore, this optimized method for psoralen-based DNA inactivation is effective to inactivate DNA in the range of 8-MOP concentrations tested and does not interfere substantially with the performance of the PCR assay.

### EXAMPLE 15

Determination of the Influence of Psoralen-Based DNA Inactivation on the Analytical Sensitivity of Three Different PCR Assays

The objective of these experiments was to determine if DNA inactivation by psoralen and UV treatment has an influence on the efficiency of three PCR assays based on fluorescence detection targetting S. agalactiae, MRSA or the genus Staphylococcus.

Method: This evaluation was performed using the SARM-specific assay described in Example 4, the S. agalactiae-specific assay described in Example 2, and the Staphylococcus-specific assay described in Example 9 except that the universal primers were not used. Purified genomic DNA was prepared as described in Example 1. A volume of 168 μL of PCR reaction mixture containing no target DNA and 0.06 μg/μL of 8-MOP was treated with UV lamps generating a wavelengths range of 320 to 400 nm and an energy of 1500 mJ/cm² (measured by the UV sensor of the Spectrolinker apparatus as described in Example 1). Sensitivity assays were performed by adding purified target genomic DNA to 25.5 μL aliquots of the treated reaction mixture. The numbers of genome copies per PCR reaction tested were 2.5, 5 and 10. There was 2 negative control reactions to which no target DNA was added. The performance of the assay was monitored by verifying three parameters including the analytical sensitivity of the assay, the cycle thresholds and the fluorescence end points.

Results and discussion: For all three fluorescence-based PCR assays evaluated there was no substantial decrease in their performance by the psoralen-based treatment as compared with an untreated reaction mixture (i.e. no 8-MOP and no UV) (FIGS. 12 and 13; Table 4). More precisely, the DNA inactivation method (i) did not influenced at all the analytical sensitivity of the three assays, (ii) increased the cycle tresholds by about 0 to 3 depending on the assay, and (iii) decreased the fluorescence end points by up to about 50%. The negative effect of the psoralen-based treatment was more important on the MRSA-specific assay (average cycle treshold increase of 1.8 (or 4.9%) and average decrease in fluorescence end points of about 46%) as compared to the S.agalactiae-specific assay (no change in the average cycle treshold and average decrease in fluorescence end points of about 17%) or the Staphylococcus-specific assay (average cycle treshold increase of 0.9 (or 2.5%) and average decrease in fluorescence end points of about 28%) (Table 4). The different composition of the reaction mixture for each PCR assay may explain this variable detrimental effect by the nucleic acid inactivation method. In conclusion, the practice of this invention yielded an optimized method for psoralen-based DNA inactivation which do not interfere substantially with the overall performance of different PCR assays. The negative influence on the performance of different PCR assays varied but remained minimal.

The conditions found above to be optimal for bNA inactivation provide for a standard to which any other system components (tubes and UV treatment components) may be compared to in order to find equivalently good inactivation. Therefore, any method, and any reagent or container comprising the reagent which result from such any method, should provide an equivalent or comparable decontamination to the following: a treatment conducted as described in Example 1 with a Spectrolinker™XL-1000 apparatus, equipped with a UV sensor and a UV source of a wavelength spectrum of about 300 to 400 nm, and providing a total energy of about 750 to 4500 mJoules per square centimeter as measured by the UV sensor located at about 17.6 cm of the UV source while a reagent is disposed in 0.6 ml MaxyClear flip cap conical plastic tubes purchased from Axygen, located at about 10.8 cm from the UV source.

It is obvious for a person skilled in the art that the UV energy values mentionned in this invention are related to the relative disposition of the reaction mixture tubes to be treated, the UV lamps and the UV sensor. A redisposition of these three elements is possible and would also fall within the scope of this invention. The energy values would need to be readjusted in accordance with the well-known laws of physics. Ideally, the sensor and the reaction mixture would need to be as close as possible from each other so that the energy measured by the sensor is very close to the energy dose really administered to the reaction mixture.

Although the present invention has been described hereinabove by way of preferred embodiments thereof, it can be modified, without departing from the spirit and nature of the subject invention as defined in the appended claims.

TABLE 1
DNA inactivation performance with the MRSA-specific
assay based on cycle treshhold analysis of UV treated
versus untreated reaction mixtures containing 8-MOP

| | Not treated with UV | | UV-treated | |
|---|---|---|---|---|
| UV lamp intensity | Cycle | Standard | Cycle | Standard |
| (μW/cm²) | threshold | deviation | threshold | deviation |
| 4200 | 26.2 | 0.2 | 35.4 | 0.1 |
| 3700 | 26.8 | 0.0 | 35.6 | 0.3 |
| 3200 | 26.4 | 0.3 | 37.9 | 0.2 |
| 2600 | 26.9 | 0.5 | 36.9 | 0.4 |
| 1900 | 26.3 | 0.1 | 36.5 | 0.2 |
| 1300 | 27.3 | 0.0 | 40.4 | 1.8 |

TABLE 2
Optimal conditions and performance for DNA inactivation
using different furocoumarins

| | | | Optimal | | Genome |
|---|---|---|---|---|---|
| | Range of | Range of | furocoumarin | | copies |
| | concentrations | UV dose tested | concentration | Optimal UV | detected after |
| Furocoumarin | tested (μg/μL)* | (mJ/cm²) | (μg/μL) | dose (mJ/cm²) | treatment |
| 8-MOP | 0.015 to 0.24 | 750 to 6000 | 0.06 | 1500 | 1 |
| Angelicin | 0.015 to 0.12 | 500 to 1500 | 0.03 | 1500 | >100 |
| 4-aminomethyltrioxsalen | 0.004 to 0.12 | 500 to 1500 | 0.004 | 750 | >100 |
| Trioxsalen | 0.001 to 0.12 | 200 to 1500 | 0.003 | 500 | 1 |
| Psoralen | 0.004 to 0.12 | 500 to 1500 | 0.004 | 750 | 10 |
| FQ (1,4,6,8-tetramethyl- | 0.003 to 0.012 | 200 to 1500 | 0.006 | 1500 | 100 |
| 2H-furo[2,3-h]quinolin-2- | | | | | |
| one) | | | | | |
| HFQ (4,6,8,9-tetramethyl- | 0.003 to 0.024 | 200 to 1500 | 0.012 | 1500 | >100 |
| 2H-furo[2,3-h]quinolin-2- | | | | | |
| one) | | | | | |

*All furocoumarins compounds were resuspended and diluted in DMSO. The final concentration of DMSO in the PCR reactions was 2.4%.

TABLE 3
Effect of DNA inactivation using various
concentrations of 8-MOP on the
performance of the MRSA-specific assay

| | Number of S. aureus genome copies per PCR reaction | | | | | |
|---|---|---|---|---|---|---|
| 8-MOP | 2.5 copies | | 5 copies | | 10 copies | |
| concentration | Cycle | Standard | Cycle | Standard | Cycle | Standard |
| (μg/μL) | threshold | deviation | threshold | deviation | threshold | deviation |
| 0 | 38.8 | 1.5 | 38.0 | 0.3 | 36.8 | 0.2 |
| 0.015 | 40.2 | 0.4 | 41.1 | 0.5 | 39.1 | 1.0 |
| 0.03 | 43.0 | 1.1 | 40.5 | 0.2 | 40.4 | 0.0 |
| 0.06 | 42.2 | 0.9 | 41.8 | 0.1 | 40.2 | 0.8 |
| 0.12 | 42.6 | 0.1 | 43.1 | 2.0 | 42.0 | 1.0 |

TABLE 4
Effect of DNA inactivation* on the performance
of three PCR assays

| | Number of | Average cycle threshold | | | Average fluorescence end point | | |
|---|---|---|---|---|---|---|---|
| | genome copies | No 8-MOP | 8-MOP | | No 8-MOP | 8-MOP | |
| PCR assay | per PCR reaction | no UV | no UV | 8-MOP + UV | no UV | no UV | 8-MOP + UV |
| MRSA- | 2.5 copies | 40.0 | 38.5 | 41.6 | 190.5 | 110.5 | 81.5 |
| specific | 5 copies | 37.6 | 37.3 | 38.9 | 162.5 | 132.5 | 110.0 |
| assay | 10 copies | 35.7 | 37.9 | 38.8 | 236.0 | 167.0 | 117.5 |
| S. agalactiae- | 2.5 copies | 43.3 | 41.8 | 42.6 | 65.5 | 55.5 | 54.5 |
| specific | 5 copies | 41.2 | 41.7 | 40.5 | 72.5 | 63.0 | 72.0 |
| assay | 10 copies | 39.7 | 40.0 | 40.7 | 93.0 | 81.0 | 63.5 |
| Staphylococcus- | 2.5 copies | 37.7 | 36.7 | 37.6 | 185.5 | 194.0 | 139.0 |
| specific | 5 copies | 35.8 | 36.7 | 36.7 | 214.5 | 188.0 | 163.0 |
| assay | 10 copies | 34.3 | 35.1 | 36.1 | 266.0 | 222.0 | 170.5 |

*The nucleic acid inactivation was performed with 0.06 μg/μL of 8-MOP and a UV treatment of 1500 mJ/cm².

TABLE 5
Effect of the UV energy dose on the performance of
DNA inactivation using 8-MOP

| | | Sensitivity | | | | |
|---|---|---|---|---|---|---|
| | | Number of | | | | |
| | | genome copies | | | | |
| | | per PCR reaction | | | DNA inactivation | |
| UV dose (mJ/cm²) | Cycle threshold | 2.5 | 5 | 10 | Untreated³ | Treated |
| 0 | Cycle threshold average | 41.8 | 40.1 | 40.0 | 25.6 | NA⁴ |
| | Standard deviation | 0.7 | 1.4 | 0.2 | 0.3 | NA |
| 750 | Cycle threshold average | 43.8 | 43.6 | 41.6 | 25.7 | 32.5 |
| | Standard deviation | 1.7 | 1.5 | 0.4 | 0.2 | 0.3 |
| 1500 | Cycle threshold average | 45.4 | 42.6 | 41.6 | 26.3 | 34.5 |
| | Standard deviation | 1.0 | 0.3 | 0.3 | 0.1 | 0.3 |
| 3000 | Cycle threshold average | 44.8 | 45.0 | 42.9 | 25.6 | 36.3 |
| | Standard deviation | 0.7 | 0.3 | 1.0 | 0.3 | 0.1 |
| 4500 | Cycle threshold average | 47.2¹ | 45.6 | 44.9 | 25.9 | 38.4 |
| | Standard deviation | NA | 0.4 | 0.0 | 0.0 | 0.4 |
| 6000 | Cycle threshold average | —² | —² | 47.5¹ | 25.9 | 43.3 |
| | Standard deviation | NA | NA | NA | 0.2 | 0.7 |

¹One out of two PCR reactions was positive.
²A dash means that both PCR reactions were negative.
³The untreated reaction mixture for the UV dose of 0 mJ/cm² contained no 8-MOP while the untreated reaction mixtures for the UV doses of 750 to 6000 mJ/cm² contained 0.06 μg/μL of 8-MOP.
⁴NA means not applicable.

1. A reagent to be put in contact with nucleic acids of interest, said reagent having a treatable surface, wherein the concentration of amplifiable contaminating nucleic acids is below a level that interferes with an amplification and/or detection reaction conducted with said nucleic acids of interest, said reagent comprising a furocoumarin compound and having been submitted to a UV light treatment capable of reducing contaminating nucleic acids below said level, with the standardization of the wavelength spectrum of the UV source and the total energy of the treatment per unit of surface, the combination of furocoumarin and UV light treatment inactivating the contaminating nucleic acids by rendering them unamplifiable;
said treatment having no substantial detrimental effect on the performance of said amplification and/or detection reaction.

2. A reagent as defined in claim 1, which is obtainable by a UV light treatment equivalent to a treatment conducted in the presence of 8-MOP as the furocoumarin, with a Spectrolinker™XL-1000 apparatus, equipped with a UV sensor and a UV source of a wavelength spectrum of about 300 to 400 nm, and providing a total energy of about 750 to 4500 mJoules per square centimeter as measured by the UV sensor located at about 17.6 cm of the UV source while a reagent is disposed in 0.6 ml MaxyClear flip cap conical plastic tubes purchased from Axygen, located at about 10.8 cm from the UV source.

3. A reagent as defined in claim 1, which is obtainable by a UV light treatment equivalent to a treatment conducted in the presence of Trioxsalen as the furocoumarin, with a Spectrolinker™ XL-1000 apparatus, equipped with a UV sensor and a UV source of a wavelength spectrum of about 300 to 400 nm, and providing a total energy of about 500 to 1500 mJoules per square centimeter as measured by the UV sensor located at about 17.6 cm of the UV source while a reagent is disposed in 0.6 ml MaxyClear flip cap conical plastic tubes purchased from Axygen, located at about 10.8 cm from the UV source.

4. A reagent as defined in claim 1, which further comprises a level of contaminating nucleic acids (either spiked or naturally present in the reagent(s)), the presence of which can be detected if its concentration is not below said level; said contaminating nucleic acids being used as a standard to monitor and optimize the conditions for nucleic acids inactivation.

5. A reagent as defined in claim 1, which comprises a protein, the function of which is not substantially affected by said treatment.

6. The reagent of claim 1, wherein said reagent comprises a component selected from the group consisting of: a nucleotide and/or nucleotide analog; an oligonucleotide primer and/or probe; a buffer solution; a monovalent and/or divalent ion; an enzyme selected from the group consisting of DNA polymerase, RNA polymerase, reverse transcriptase, DNA ligase, restriction enzyme DNAase, RNAase, protease and an enzyme used for NAT or in test sample preparation for NAT; an amplification facilitator; a cryoprotector; a stabilizer; a solvent; and any suitable combination thereof.

7. The reagent of claim 6, wherein at least two components are mixed together in a common vial.

8. The reagent of claim 1, which is liquid, frozen or dehydrated.

9. A container comprising a reagent as defined in claim 1.

10. (cancelled)

11. A container as defined in claim 9, which is a closed vessel.

12. A reagent, as defined in claim 1, wherein said furocoumarin is 8-MOP or Trioxsalen.

13. A reagent or as defined in claim 12, wherein 8-MOP is used at a final concentration of about 0.015 μg/μL (or 0.07 mM) to about 0.12 μg/μL (or 0.56 mM).

14. A reagent as defined in claim 12, wherein Trioxsalen is used at a final concentration of about 0.001 μg/μL (0.0044 mM) to 0.0075 μg/μL (0.033 mM).

15. A reagent as defined in claim 1, which is for PCR.

16. A method for rendering contaminating nucleic acids in a reagent unamplifiable in an amplification reaction of nucleic acids of interest, without substantially affecting the performance of the amplification reaction which comprises:
a) providing a reagent to be contacted with said nucleic acids of interest;
b) providing a furocoumarin compound;
c) obtaining a mixture of said reagent and the furocoumarin compound; and
d) treating said mixture with light energy of a wavelength in the UV range.

17. A method as defined in claim 16, wherein the UV light treatment is equivalent to a treatment conducted in the presence of 8-MOP as the furocoumarin, with a Spectrolinker™XL-1000 apparatus, equipped with a UV sensor and a UV source of a wavelength spectrum of about 300 to 400 nm, and providing a total energy of about 750 to 4500 mJoules per square centimeter as measured by the UV sensor located at about 17.6 cm of the UV source while a reagent is disposed in 0.6 ml MaxyClear flip cap conical plastic tubes purchased from Axygen, located at about 10.8 cm from the UV source.

18. A method as defined in claim 16, wherein said UV light treatment is equivalent to a treatment conducted in the presence of Trioxsalen as the furocoumarin, with a Spectrolinker™ XL-1000 apparatus, equipped with a UV sensor and a UV source of a wavelength spectrum of about 300 to 400 nm, and providing a total energy of about 500 to 1500 mJoules per square centimeter as measured by the UV sensor located at about 17.6 cm of the UV source while a reagent is disposed in 0.6 ml MaxyClear flip cap conical plastic tubes purchased from Axygen, located at about 10.8 cm from the UV source.

19. A method as defined in claim 16, which further comprises a level of contaminating nucleic acids, the presence of which can be detected if its concentration is not below said level; said contaminating nucleic acids being used as a standard to monitor and optimize the conditions for nucleic acids inactivation.

20. The method of claim 16, wherein the reagent comprises a protein, the function of which is not substantially affected by said treatment.

21. The method of any one of claim 16, wherein the furocoumarin compound is a psoralen or an isopsoralen derivative.

22. The method of claim 21, wherein the furocoumarin compound is 8-MOP or Trioxsalen.

23. The method of claim 21, wherein the concentration of the furocoumarin compound is about 0.015 μg/μL (or 0.07 mM) to about 0.12 μg/μL (or 0.56 mM).

24. The method of claim 21, wherein the concentration of the furocoumarin compound is about 0.001 μg/μL (or 0.0044 mM) to 0.0075 μg/μL (or 0.033 mM).

25. The method of claim 16, wherein the reagent is involved in an amplification and/or detection reaction or in the test sample preparation.

26. The method of claim 25, wherein the reagent comprises a component selected from the group consisting of: a nucleotide and/or nucleotide analog; an oligonucleotide primer and/or probe; a buffer solution; a monovalent and/or divalent ion; an enzyme selected from the group consisting of DNA polymerase, RNA polymerase, reverse transcriptase, DNA ligase, restriction enzyme, DNAase, RNAase, protease and any enzyme used for NAT or in test sample preparation for NAT; an amplification facilitator; a cryoprotector; a stabilizer; a solvent; and any suitable combination thereof.

27. The method of claim 25, wherein the reagent is a PCR reagent.

28. The method of claim 25, wherein the reagent is a RT-PCR reagent.

29. The method of claim 16 wherein said mixture is treated with UV in a tubing.

30. The method of claim 16, wherein said mixture is enclosed in a plastic vessel.

31. The method of claim 30, wherein said mixture is treated with UV in immediate container into which a reaction with nucleic acids of interest is performed.

32. The method of claim 16, wherein said UV light dose is applied and monitored by measurements with a radiometer equipped with a UV sensor or with an appropriate spectrometer.

33. The method of claim 16, wherein said reaction mixture is treated using a suitable UV source including a laser, high intensity white light, an incandescent lamp and a diode.

34. The method of claim 16, wherein the light treatment is performed using an apparatus consisting of a chamber equipped with UV lights and allowing to measure the UV dose.
